SEQUENCE LISTING (9 sequences)

SEQ ID NO: 1 - protein, 216 aa, Artificial Sequence
  Description of Artificial Sequence Synthetic polypeptide
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 2 - protein, 445 aa, Artificial Sequence
  Description of Artificial Sequence Synthetic polypeptide
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 14 aa, Artificial Sequence
  Description of Artificial Sequence Synthetic peptide
TGTGSDVGSYNLVS

SEQ ID NO: 4 - protein, 7 aa, Artificial Sequence
  Description of Artificial Sequence Synthetic peptide
GDSERPS

SEQ ID NO: 5 - protein, 10 aa, Artificial Sequence
  Description of Artificial Sequence Synthetic peptide
SSYAGSGIYV

SEQ ID NO: 6 - protein, 5 aa, Artificial Sequence
  Description of Artificial Sequence Synthetic peptide
TYAMG

SEQ ID NO: 7 - protein, 14 aa, Artificial Sequence
  Description of Artificial Sequence Synthetic peptide
SIGASGSQTRYADS

SEQ ID NO: 8 - protein, 7 aa, Artificial Sequence
  Description of Artificial Sequence Synthetic peptide
LAIGDSY

SEQ ID NO: 9 - protein, 6 aa, Artificial Sequence
  Description of Artificial Sequence Synthetic 6xHis tag
HHHHHH

(19) United States
(12) Patent Application Publication
(10) Pub. No.: US 2021/0299255 A1
(43) Pub. Date: Sep. 30, 2021

(54) COMPOSITIONS OF FCRN ANTIBODIES AND METHODS OF USE THEREOF

(71) Applicant: MOMENTA PHARMACEUTICALS, INC., Cambridge, MA (US)

(72) Inventors: Zhongli ZHANG, Cambridge, MA (US); Gregory ST. LOUIS, Cambridge, MA (US); Eva WILLIAMS, Cambridge, MA (US); Narinder SINGH, Cambridge, MA (US); Siddhesh PATIL, Cambridge, MA (US)

(21) Appl. No.: 17/260,318

(22) PCT Filed: Jul. 19, 2019
(86) PCT No.: PCT/US19/42597

§ 371 Date: Jan. 14, 2021

Related U.S. Application Data
(60) Provisional application No. 62/701,467, filed on Jul. 20, 2018.

(51) Int. Cl. A61K 39/395; C07K 16/28; A61K 47/02; A61K 47/26; A61K 47/12; A61K 9/00

(52) U.S. Cl. CPC A61K 39/39591; C07K 16/283; A61K 9/0019; A61K 47/26; A61K 47/12; A61K 47/02

(57) ABSTRACT

Stable pharmaceutical compositions containing an anti-FcRn antibody are described and characterized.

### BACKGROUND

Numerous autoimmune and alloimmune diseases are mediated by pathogenic antibodies. The stability, activity, and transport of pathogenic antibodies depends on the Fc receptor (FcRn), a type I transmembrane protein that functions as an IgG- and serum albumin-binding, intracellular vesicular trafficking protein. For example, many fetal and neonatal immune diseases result from the transfer of maternal antibodies from a pregnant subject, especially a pregnant subject with an immunological disease, to the fetus through the human neonatal Fc receptor (FcRn) in the placenta

### SUMMARY

This disclosure pertains to compositions comprising an anti-FcRn antibody (M281 compositions) and methods of using such compositions in the treatment of autoimmune diseases.

Described herein is a pharmaceutical composition includes: an antibody that includes a heavy chain includes the amino acid sequence of SEQ ID NO:2 with up to 5 single amino acid insertions, substitutions or deletions and a light chain includes the amino acid sequence of SEQ ID NO:1 with up to 5 single amino acid insertions, substitutions or deletions at 10 or 30 mg/ml, 20-30 mM sodium phosphate, 20-30 mM sodium chloride, 80-100 (e.g., 90-91 mg/ml Trehalose, and 0.1-0.005% w/v Polysorbate 80, buffered at pH 6.5).

In various cases the composition: includes 25 mM sodium phosphate; includes 25 mM sodium chloride; includes 90-91 mg/ml Trehalose; includes 90.5 mg/ml Trehalose; includes 0.01% w/v Polysorbate 80; includes 25 mM sodium phosphate, 25 mM sodium chloride, 90.5 mg/ml Trehalose, and 0.01% Polysorbate 80; the composition does not comprise any additional excipients; the composition does not include any polysorbates other than polysorbate 80, the composition does not include any polymers other than a polysorbate, the composition does not include any polymers other than polysorbate 80, the antibody comprises a heavy chain includes the amino acid sequence of SEQ ID NO:2 with up to 2 single amino acid insertions, substitutions or deletions and having a light chain includes the amino acid sequence of SEQ ID NO:1 with up to 2 single amino acid insertions, substitutions or deletions; the antibody comprises a heavy chain includes the amino acid sequence of SEQ ID NO:2 with up to 2 single amino acid substitutions and having a light chain includes the amino acid sequence of SEQ ID NO:1 with up to 2 single amino acid substitutions; the antibody comprises a heavy chain includes the amino acid sequence of SEQ ID NO:2 and a light chain includes the amino acid sequence of SEQ ID NO:1.

Also described is a pharmaceutical composition includes: an antibody includes a heavy chain includes the amino acid sequence of SEQ ID NO:24 with up to 5 single amino acid insertions, substitutions or deletions and a light chain includes the amino acid sequence of SEQ ID NO:19 with up to 5 single amino acid insertions, substitutions or deletions at 10 or 30 mg/ml, 20-30 mM sodium succinate, 20-30 mM sodium chloride, 89-92 mg/ml Trehalose, and 0.02-0.005% w/v Polysorbate 80, buffered at pH 6.5.

In various cases the composition: includes 25 mM sodium succinate; includes 25 mM sodium chloride; includes 90-91 mg/ml Trehalose; includes 90.5 mg/ml Trehalose; includes 0.01% w/v Polysorbate 80; includes 25 mM sodium succinate, 25 mM sodium chloride, 90.5 mg/ml Trehalose, and 0.01% Polysorbate 80; the composition does not comprise any additional excipients; the antibody comprises a heavy chain includes the amino acid sequence of SEQ ID NO:2 with up to 2 single amino acid insertions, substitutions or deletions and having a light chain includes the amino acid sequence of SEQ ID NO:1 with up to 2 single amino acid insertions, substitutions or deletions; the antibody comprises a heavy chain includes the amino acid sequence of SEQ ID NO:2 with up to 2 single amino acid substitutions and having a light chain includes the amino acid sequence of SEQ ID NO:19 with up to 2 single amino acid substitutions; the antibody comprises a heavy chain includes the amino acid sequence of SEQ ID NO:2 and having a light chain includes the amino acid sequence of SEQ ID NO:1.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF THE DRAWINGS

FIG. 1 shows data for thermal transitions as measured by DSC for formulations in Table 1

FIG. 2 shows data for size purity as measured by SEC at accelerated conditions for formulations in Table 1.

FIG. 3 shows data for charge heterogeneity as measured by cIEF at accelerated conditions for formulations in Table 1.

FIG. 4 shows data for purity as measured by CE-SDS Caliper (Non-Reduced) at accelerated conditions for formulations in Table 1.

FIG. 5 shows data for purity as measured by CE-SDS Caliper (Reduced) at accelerated conditions for formulations in Table 1.

FIG. 6 shows data for size purity as measured by SEC at accelerated conditions for formulations in Table 2.

FIG. 7 shows data for charge heterogeneity as measured by cIEF at accelerated conditions for formulations in Table 2.

FIG. 8 shows data for size distribution as measured by Dynamic light scattering (DLS) for formulations in Table 2.

FIG. 9 shows data for purity as measured by CE-SDS Caliper (Non-Reduced) at accelerated conditions for formulations in Table 2.

FIG. 10 shows data for purity as measured by CE-SDS Caliper (Reduced) at accelerated conditions for formulations in Table 2.

FIG. 11 shows data for thermal transitions as measured by DSC for various buffer pHs for formulations in Table 2. Higher Tm onset indicates better thermal stability of the protein at the particular pH. Three transitions were identified in the pH screening study, Tm1, Tm2, and Tm3.

FIG. 12 shows a comparison of antibody % Main Species Levels by cIEF under temperature stress. Antibody stability results at long term storage conditions of 2-8° C. are shown as a solid line, and accelerated storage conditions of 25° C./60% RH as dotted line. Anitibody at 30 mg/mL for Lot D is shown in red. Antibody at 10 mg/mL for Lot E is shown in black, for Lot F shown in purple, and for Lot B shown in blue. Note: The green specification line applies to real time conditions at 2-8° C. only.

FIG. 13 shows a comparison of antibody % Main Species Levels by SEC-HPLC under temperature stress. Antibody stability results at long term storage conditions of 2-8° C. are shown as a solid line, and accelerated storage conditions of 25° C./60% RH as dotted line. Antibody at 30 mg/mL for Lot D is shown in red. Anitbody at 10 mg/mL for Lot E is shown in black, for Lot F shown in purple, and for Lot B shown in blue. The green specification line applies to real time conditions at 2-8° C. only.

FIGS. 14A-14D shows data for protein concentration of 10 mg/mL DP development Lot E through 30 months, Lot F through 24 months, GMP Lot A through 24 months, GMP Lot B through 18 months (A) at long term storage condition 2-8° C. in stability study and the regression study plot for all the 10 mg/mL DP lots (B); concentration of 30 mg/mL development DP Lot D through 12 months and GMP Lot C through 3 months at long term storage condition 2-8° C. in stability study (C) and the regression study plot for DP Lot D (D). USL: upper specification limit; LSL: lower specification limit

FIGS. 15A-15D shows pH of 10 mg/mL DP development Lot E through 30 months, Lot F through 24 months, GMP Lot A through 24 months, Lot B through 18 months (A) at long term storage condition 2-8 OC in stability study and the regression study plot for all the 10 mg/mL DP lots (B); pH of development DP Lot D through 12 months and GMP Lot C through 3 months at long term storage condition 2-8 OC in stability study (C) and the regression study plot for DP Lot D (D). USL: upper specification limit; LSL: lower specification limit.

FIGS. 16A-16D shows data for size purity of 10 mg/mL DP development Lot E through 30 months, Lot F through 24 months, GMP Lot A through 24 months, Lot B through 18 months (A) at long term storage condition 2-8 OC in stability study and the regression study plot for all the 10 mg/mL DP lots (B); Size purity by SEC of development DP Lot D through 12 months and GMP Lot C through 3 months at long term storage condition 2-8 OC in stability study (C) and the regression study plot for DP Lot D (D). LSL: lower specification limit.

FIGS. 17A-17D shows data for purity by reduced CE-SDS of 10 mg/mL DP development Lot E through 30 months, Lot F through 24 months, GMP Lot A through 24 months, Lot B through 18 months (A) at long term storage condition 2-8 OC in stability study and the regression study plot for all the 10 mg/mL DP lots (B); HC+LC purity by reduced CE-SDS of development DP Lot D through 12 months and GMP Lot C through 3 months at long term storage condition 2-8 OC in stability study (C) and the regression study plot for DP Lot D (D). LSL: lower specification limit.

FIGS. 18A-18D shows data for size purity by non-reduced CE-SDS of 10 mg/mL DP development Lot E through 30 months, Lot F through 24 months, GMP Lot A through 24 months, Lot B through 18 months (A) at long term storage condition 2-8 OC in stability study and the regression study plot for all the 10 mg/mL DP lots (B); size purity by non-reduced CE-SDS of development DP Lot D through 12 months and GMP Lot C through 3 months at long term storage condition 2-8 OC in stability study (C) and the regression study plot for DP Lot D (D). LSL: lower specification limit.

FIGS. 19A-D shows data for peak A level by non-reduced CE-SDS of 10 mg/mL DP development Lot E through 30 months, Lot F through 24 months, GMP Lot A through 24 months, Lot B through 18 months (A) at long term storage condition 2-8 OC in stability study and the regression study plot for all the 10 mg/mL DP lots (B); Peak A level by non-reduced CE-SDS of development DP Lot D through 12 months and GMP Lot C through 3 months at long term storage condition 2-8 OC in stability study (C) and the regression study plot for DP Lot D (D). USL: upper specification limit.

FIGS. 20 A-D shows data for the main peak from cIEF of 10 mg/mL DP development Lot E through 30 months, Lot F through 24 months, GMP Lot A through 24 months, Lot B through 18 months (A) at long term storage condition 2-8 OC in stability study and the regression study plot for all the 10 mg/mL DP lots (B); Main peak from cIEF of development DP Lot D through 12 months and GMP Lot C through 3 months at long term storage condition 2-8 OC in stability study (C) and the regression study plot for DP Lot D (D). LSL: lower specification limit.

FIGS. 21A-21D shows data for the acidic peak from cIEF of 10 mg/mL DP development Lot E through 30 months, Lot F through 24 months, GMP Lot A through 24 months, Lot B through 18 months (A) at long term storage condition 2-8 OC in stability study and the regression study plot for all the 10 mg/mL DP lots (B); Acidic peak from cIEF of development DP Lot D through 12 months and GMP Lot C through 3 months at long term storage condition 2-8 OC in stability study (C) and the regression study plot for DP Lot D (D). USL: upper specification limit.

FIGS. 22A-22D shows data for the Basic peak from cIEF of 10 mg/mL DP development Lot E through 30 months, Lot F through 24 months, GMP Lot A through 24 months, Lot B through 18 months (A) at long term storage condition 2-8 OC in stability study and the regression study plot for all the 10 mg/mL DP lots (B); Basic peak from cIEF of development DP Lot D through 12 months and GMP Lot C through 3 months at long term storage condition 2-8 OC in stability study (C) and the regression study plot (D). USL: upper specification limit.

FIGS. 23A-23D shows the data for potency of 10 mg/mL DP GMP Lot A through 24 months, Lot B through 18 months (A) at long term storage condition 2-8 OC in stability study and the regression study plot for both 10 mg/mL DP lots (B); Potency of development DP Lot D through 12 months and GMP Lot C through 3 months at long term storage condition 2-8 OC in stability study (C) and the regression study plot (D). USL: upper specification limit; LSL: lower specification limit.

### DETAILED DESCRIPTION

The present disclosure features compositions comprising antibodies to human neonatal Fc receptor (FcRn). These compositions are useful, e.g., to promote clearance of autoantibodies in a subject, to suppress antigen presentation in a subject, to block an immune response, e.g., block an immune complex-based activation of the immune response in a subject, or to treat immunological diseases (e.g., autoimmune diseases) in a subject.

Following initial studies, select formulations were prepared with different concentrations of sodium chloride, Trehalose, and surfactant polysorbate (PS) 80, buffered agents and buffered at different pH (pH 5 to 8). Thus, the compositions include both an ionic osmolyte stabilizer (sodium chloride) and non-ionic osmolyte stabilizer (trehalose) The stability of the aforementioned formulations was assessed over time by appearance, pH, protein concentration, size purity, charge distribution, and thermal stability. These stability parameters were measured by analytical techniques including pH, UV-Vis, size exclusion chromatography, ion exchange chromatography, CE-SDS, and differential scanning calorimetry.

Two formulations exhibited enhanced stability as assessed across the aforementioned metrics and the stability was sustained over time: (1) 25 mM sodium phosphate, 25 mM sodium chloride, 90.5 mg ml⁻¹ Trehalose, 0.01% polysorbate (PS) 80, and antibody (having heavy chain comprising sequence SEQ ID NO:2 and a light chain comprising SEQ ID NO:1) at 10 or 30 mg ml⁻¹ buffered at pH 6.5; and (2) 25 mM sodium succinate, 25 mM sodium chloride, 90.5 mg ml⁻¹ Trehalose, 0.01% polysorbate (PS) 80, and antibody (having heavy chain comprising sequence SEQ ID NO:2 and a light chain comprising SEQ ID NO:1) at 10 or 30 mg ml⁻¹ buffered at pH 6.6. The stability of the aforementioned two formulations was further tested in presence of select mechanical, thermal, and chemical stresses. Both formulations exhibited no significant deterioration in stability as assessed across the multiple aforementioned metrics over time. Notably the stability was maintained for more than 30 months for the formulation (1) 25 mM sodium phosphate, 25 mM sodium chloride, 90.5 mg ml-1 Trehalose, 0.01% polysorbate (PS) 80, and antibody at 10 or 30 mg ml-1 buffered at pH 6.5. Also tested was a formulation that 25 mM sodium phosphate, 25 mM sodium chloride, 90.5 mg ml-1 Trehalose, and antibody (having heavy chain comprising sequence SEQ ID NO:2 and a light chain comprising SEQ ID NO:1) buffered at pH 6.5 with differing amounts of polysorbate 80.

### Anti-FcRn Antibodies

Antibodies that can be formulated as described herein include an antibody having the light chain sequence of SEQ ID NO:1 and the heavy chain sequence of SEQ ID NO:2 (also referred to as M281; compositions containing this antibody are sometimes referred to as M281 compositions. Variants of this antibody can also be formulated as described herein. Such variants include: an antibody having a light chain sequence of a variant of SEQ ID NO:1 having 1-5 single amino acid substitution or deletions (and preferably comprising the CDR sequences of SEQ ID Nos: 3-5) and a heavy chain sequence of a variant of SEQ ID NO:2 having 1-5 single amino acid substitution or deletions (and preferably comprising the CDR sequences of SEQ ID Nos: 6-8). Antibodies that are composed of a variant of SEQ ID NO:1 and a variant of SEQ ID NO:2, preferably retain the CDR sequences: TGTGSDVGSYNLVS (light chain CDR1; SEQ ID NO: 3); GDSERPS (light chain CDR2; SEQ ID NO: 4); SSYAGSGIYV (light chain CDR3; SEQ ID NO: 5); TYAMG (heavy chain CDR1; SEQ ID NO: 6); SIGASGSQTRYADS (heavy chain CDR2; SEQ ID NO: 7); and LAIGDSY (heavy chain CDR3; SEQ ID NO: 8).

In some cases, the light chain has a sequence having at least 90%, 95% or 98% identity:

(SEQ ID NO: 1)
QSALTQPASVSGSPGQSITISCTGTGSDVGSYNLVSWYQQHPGKAPKLMI
YGDSERPSGVSNRFSGSKSGNTASLTISGLQAEDEADYYCSSYAGSGIYV
FGTGTKVTVLGQPKAAPSVTLFPPSSEELQANKATLVCLISDFYPGAVTV
AWKADSSPVKAGVETTTPSKQSNNKYAASSYLSLTPEQWKSHKSYSCQVT
HEGSTVEKTVAPTECS.

In some cases, the heavy chain has a sequence having at least 90%, 95%, or 98% identity to:

(SEQ ID NO: 2)
EVQLLESGGGLVQPGGSLRLSCAASGFTFSTYAMGWVRQAPGKGLEWVSS
IGASGSQTRYADSVKGRFTISRDNSKNTLYLQMNSLRAEDTAVYYCARLA
IGDSYWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYASTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG.

### Vectors, Host Cells, and Antibody Production

Anti-FcRn antibodies can be produced from a host cell. A host cell refers to a vehicle that includes the necessary cellular components, e.g., organelles, needed to express the polypeptides and constructs described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, etc). The choice of nucleic acid vectors depends in part on the host cells to be used. Generally, preferred host cells are of either prokaryotic (e.g., bacterial) or eukaryotic (e.g., mammalian) origin.

### Nucleic Acid Vector Construction and Host Cells

A nucleic acid sequence encoding the amino acid sequence of an anti-FcRn antibody may be prepared by a variety of methods known in the art. These methods include, but are not limited to, oligonucleotide-mediated (or site-directed) mutagenesis and PCR mutagenesis. A nucleic acid molecule encoding an anti-FcRn antibody may be obtained using standard techniques, e.g., gene synthesis. Alternatively, a nucleic acid molecule encoding a wild-type anti-FcRn antibody may be mutated to contain specific amino acid substitutions using standard techniques in the art, e.g., QuikChange™ mutagenesis. Nucleic acid molecules can be synthesized using a nucleotide synthesizer or PCR techniques.

Nucleic acid sequences encoding an anti-FcRn antibody may be inserted into a vector capable of replicating and expressing the nucleic acid molecules in prokaryotic or eukaryotic host cells. Many vectors are available in the art and can be used. Each vector may contain various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

Mammalian cells can be used as host cells. Examples of mammalian cell types include, but are not limited to, human embryonic kidney (HEK) (e.g., HEK293, HEK 293F), Chinese hamster ovary (CHO), HeLa, COS, PC3, Vero, MC3T3, NSO, Sp2/0, VERY, BHK, MDCK, W138, BT483, Hs578T, HTB2, BT20, T47D, NSO (a murine myeloma cell line that does not endogenously produce any immunoglobulin chains), CRL7O3O, and HsS78Bst cells. In other can, E. coli cells can be used as host cells. Examples of E. coli strains include, but are not limited to, E. coli 294 (ATCC® 31,446), E. coli λ 1776 (ATCC® 31,537, E. coli BL21 (DE3) (ATCC® BAA-1025), and E. coli RV308 (ATCC® 31,608). Different host cells have characteristic and specific mechanisms for the posttranslational processing and modification of protein products. Appropriate cell lines or host systems may be chosen to ensure the correct modification and processing of the anti-FcRn antibody expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 (Jul. 20, 2004) and Vladimir Voynov and Justin A. Caravella (eds.) Therapeutic Proteins: Methods and Protocols (Methods in Molecular Biology) Humana Press; 2nd ed. 2012 (Jun. 28, 2012).

### Protein Production, Recovery, and Purification

Host cells used to produce an anti-FcRn antibody may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin Host cells are cultured at suitable temperatures, such as from about 20° C. to about 39° C., e.g., from 25° C. to about 37° C., preferably 37° C., and CO₂ levels, such as 5 to 10% (preferably 8%). The pH of the medium is generally from about 6.8 to 7.4, e.g., 7.0, depending mainly on the host organism. If an inducible promoter is used in the expression vector, protein expression is induced under conditions suitable for the activation of the promoter.

Protein recovery typically involves disrupting the host cell, generally by such means as osmotic shock, sonication, or lysis. Once the cells are disrupted, cell debris may be removed by centrifugation or filtration. The proteins may be further purified. An anti-FcRn antibody may be purified by any method known in the art of protein purification, for example, by protein A affinity, other chromatography (e.g., ion exchange, affinity, and size-exclusion column chromatography), centrifugation, differential solubility, or by any other standard technique for the purification of proteins. (see Process Scale Purification of Antibodies, Uwe Gottschalk (ed.) John Wiley & Sons, Inc., 2009). In some instances, an anti-FcRn antibody can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from the influenza hemagglutinin protein.

### Methods of Treatment and Indications

The blockade of human FcRn by the pharmaceutical compositions containing anti-FcRn antibodies described herein may be of therapeutic benefit in diseases that are driven by IgG autoantibodies. The ability of FcRn blockade to induce overall IgG catabolism and removal of multiple species of autoantibodies, small circulating metabolites, or lipoproteins offers a method to expand the utility and accessibility of an autoantibody removal strategy to patients with autoantibody-driven autoimmune disease pathology. Without being bound any theory, the dominant mechanism of action of an anti-FcRn antibody may be to increase the catabolism of pathogenic autoantibodies in circulation and decrease autoantibody and immune complex deposition in affected tissues.

The pharmaceutical compositions are useful to promote catabolism and clearance of pathogenic antibodies, e.g., IgG and IgG autoantibodies in a subject, to reduce the immune response, e.g., to block immune complex-based activation of the immune response in a subject, and to treat immunological conditions or diseases in a subject. In particular, the pharmaceutical compositions are useful to reduce or treat an immune complex-based activation of an acute or chronic immune response. The acute immune response may be activated by a medical condition selected from the group consisting of pemphigus vulgaris, lupus nephritis, myasthenia gravis, Guillain-Barré syndrome, antibody-mediated rejection, catastrophic anti-phospholipid antibody syndrome, immune complex-mediated vasculitis, glomerulitis, a channelopathy, neuromyelitis optica, autoimmune hearing loss, idiopathic thrombocytopenia purpura (ITP), autoimmune haemolytic anaemia (AIHA), immune neutropenia, dialated cardiomyopathy, and serum sickness. The chronic immune response may be activated by a medical condition selected from the group consisting of chronic inflammatory demyelinating polyneuropathy (CIDP), systemic lupus, a chronic form of a disorder indicated for acute treatment, reactive arthropathies, primary biliary cirrhosis, ulcerative colitis, and antineutrophil cytoplasmic antibody (ANCA)-associated vasculitis.

In some cases, the pharmaceutical compositions are useful to reduce or treat an immune response activated by an autoimmune disease. The autoimmune disease may be selected from the group consisting of alopecia areata, ankylosing spondylitis, antiphospholipid syndrome, Addison's disease, hemolytic anemia, autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Ménière's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, antineutrophil cytoplasmic antibody (ANCA)-associated vasculitis, myasthenia gravis, neuromyelitis optica or Wegener's granulomatosis.

In some cases, the pharmaceutical compositions are useful to decrease the risk of or decrease the risk of developing anemia in the fetus. In some cases, the pharmaceutical compositions are useful to decrease or obviate the need for IUT (intrauterine transfusion). In some cases, the pharmaceutical compositions and methods are useful to decrease or obviate the need for antenatal PP+IVIg, postnatal transfusion, IVIg, and/or phototherapy.

In some cases, the pharmaceutical compositions are useful to reduce or treat an immune response in a fetus or neonate. In some cases, the pharmaceutical compositions and methods are useful to reduce or treat an immune response in a fetus or neonate activated by an autoimmune disease in the pregnant mother.

In particular, the pharmaceutical compositions are useful to reduce or treat an immune response activated by systemic lupus erythematosus, antiphospholipid syndrome, pemphigus vulgaris/bullous pemphigoid, antineutrophil cytoplasmic antibody (ANCA)-associated vasculitis, myasthenia gravis, or neuromyelitis optica. In some cases, the pharmaceutical compositions are useful to reduce or treat an immune response in a fetus or neonate. In some cases, the pharmaceutical compositions and methods are useful to reduce or treat an immune response activated by systemic lupus erythematosus, antiphospholipid syndrome, pemphigus vulgaris/bullous pemphigoid, antineutrophil cytoplasmic antibody (ANCA)-associated vasculitis, myasthenia gravis, or neuromyelitis optica in the pregnant mother.

The pharmaceutical compositions are useful in methods of decreasing pathogenic antibody transport (e.g., pathogenic maternal IgG antibody transport) across the placenta of a pregnant subject, increasing pathogenic antibody catabolism in a pregnant subject, and treating an antibody-mediated enhancement of viral disease in a fetus or a neonate by administering to a pregnant subject an isolated antibody that binds to human FcRn. Diseases and disorders that may benefit from FcRn inhibition by the pharmaceutical compositions described herein include diseases and disorders in a fetus and/or neonate that are caused by the transfer of maternal pathogenic antibodies (e.g., maternal pathogenic IgG antibodies) across the placenta from a pregnant subject to the fetus and/or neonate.

In some cases, the diseases and disorders that may benefit from treatment with the pharmaceutical compositions described herein are fetal and neonatal alloimmune and/or autoimmune disorders. Fetal and neonatal alloimmune disorders are disorders in a fetus and/or neonate that is caused by pathogenic antibodies in the pregnant subject. The pathogenic antibodies in the pregnant subject may attack the antigens of the fetus (e.g., antigens the fetus inherited from the fetus' father), causing the fetus or the neonate to have a fetal and neonatal alloimmune and/or autoimmune disorder.

Examples of fetal and neonatal alloimmune and/or autoimmune disorders that may be treated include, but are not limited to, fetal and neonatal alloimmune thrombocytopenia (FNAIT), hemolytic disease of the fetus and newborn (HDFN), alloimmune pan-thrombocytopenia, congenital heart block, fetal arthrogryposis, neonatal myasthenia gravis, neonatal autoimmune hemolytic anemia, neonatal anti-phospholipid syndrome, neonatal polymyositis, dermatomyositis, neonatal lupus, neonatal scleroderma. Behcet's disease, neonatal Graves' disease, neonatal Kawasaki disease, neonatal autoimmune thyroid disease, and neonatal type I diabetes mellitus.

In some cases, the diseases and disorders that may benefit from treatment with the pharmaceutical compositions described herein are viral diseases wherein antibodies facilitate viral entry into host cells, leading to increased or enhanced infectivity in the cells, e.g., antibody-mediated enhancement of viral disease. In some cases, an antibody may bind to a viral surface protein and the antibody/virus complex may bind to an FcRn on a cell surface through interaction between the antibody and the receptor. Subsequently, the antibody/virus complex may get internalized into the cell. For example, a virus may gain entry into the cells and/or tissues of a fetus through forming a complex with a maternal IgG antibody. A maternal IgG antibody may bind to a viral surface protein and the IgG/virus complex may bind to an FcRn in the syncytiotrophoblasts of the placenta, which then transfers the complex into the fetus.

In some cases, the pharmaceutical compositions described herein may be used to treat an antibody-mediated enhancement of viral disease. In some cases, the viral diseases that are enhanced by pathogenic antibodies (e.g., pathogenic IgG antibodies) include, but are not limited to, viral diseases caused by an alpha virus infection, flavivirus infection, Zika virus infection, Chikungunya virus infection, Ross River virus infection, severe acute respiratory syndrome coronavirus infection, Middle East respiratory syndrome, avian influenza infection, influenza virus infection, human respiratory syncytial virus infection, Ebola virus infection, yellow fever virus infection, dengue virus infection, human immunodeficiency virus infection, respiratory syncytial virus infection, Hantavirus infection, Getah virus infection, Sindbis virus infection, Bunyamwera virus infection, West Nile virus infection, Japanese encephalitis virus B infection, rabbitpox virus infection, lactate dehydrogenase elevating virus infection, reovirus infection, rabies virus infection, foot-and-mouth disease virus infection, porcine reproductive and respiratory syndrome virus infection, simian hemorrhagic fever virus infection, equine infectious anemia virus infection, caprine arthritis virus infection, African swine fever virus infection, lentivirus infection, BK papovavirus infection, Murray Valley encephalitis virus infection, enterovirus infection, cytomegalovirus infection, pneumovirus infection, morbillivirus infection, and measles virus infection.

The blockade of human FcRn by anti-FcRn antibodies may be of therapeutic benefit in diseases that are driven by pathogenic antibodies (e.g., pathogenic IgG antibodies). The ability of FcRn blockade to induce overall pathogenic antibody catabolism and removal of multiple species of pathogenic antibodies without perturbing serum albumin, small circulating metabolites, or lipoproteins offers a method to expand the utility and accessibility of a pathogenic antibody removal strategy to patients with pathogenic antibody-driven autoimmune disease pathology. While not bound by theory, the dominant mechanism of action of an anti-FcRn antibody may be to increase the catabolism of pathogenic antibodies in circulation and decrease pathogenic antibody and immune complex deposition in affected tissues.

The pharmaceutical compositions described herein may be administered to a pregnant subject who has or is at risk of having a medical condition that activates an immune response in the pregnant subject. In some cases, the pregnant subject may have had, in the past, a medical condition that activated an immune response in the pregnant subject. In some cases, the pregnant subject has a history of having had a previous fetus or neonate that had a fetal and neonatal alloimmune and/or autoimmune disorder. In some cases, the anti-FcRn antibodies described herein may be administered to a pregnant subject if a pathogenic antibody associated with an immune disease is detected in a biological sample (e.g., a blood or urine sample) obtained from the pregnant subject. In some cases, the pathogenic antibody detected in the biological sample of the pregnant subject is known to bind to an antigen from the fetus in the pregnant subject (e.g., an antigen that the fetus inherited from the fetus' father).

In some cases, the pharmaceutical compositions may be administered to a subject who is planning to become pregnant and who has or is at risk of having a medical condition that activates an immune response in the pregnant subject, and/or who has had, in the past, a medical condition that activated an immune response in the pregnant subject. In some cases, a subject is planning to become pregnant and has a history of having had a previous fetus or neonate that had a fetal and neonatal alloimmune and/or autoimmune disorder. In some cases, the anti-FcRn antibodies described herein may be administered to a subject who is planning to become pregnant and whose biological sample contains a pathogenic antibody associated with an immune disease.

In some cases, the pharmaceutical compositions described herein may be administered to a subject (e.g., a pregnant subject) to reduce or treat an immune complex-based activation of an acute or chronic immune response in the subject. The acute immune response may be activated by a medical condition (e.g., pemphigus vulgaris, lupus nephritis, myasthenia gravis, Guillain-Barré syndrome, antibody-mediated rejection, catastrophic anti-phospholipid antibody syndrome, immune complex-mediated vasculitis, glomerulitis, a channelopathy, neuromyelitis optica, autoimmune hearing loss, idiopathic thrombocytopenia purpura, autoimmune haemolytic anaemia, immune neutropenia, dialated cardiomyopathy, serum sickness, chronic inflammatory demyelinating polyneuropathy, systemic lupus, reactive arthropathies, primary biliary cirrhosis, ulcerative colitis, or antineutrophil cytoplasmic antibody (ANCA)-associated vasculitis).

In some cases, the formulation described herein may be administered to a subject (e.g., a pregnant subject) to reduce or treat an immune response activated by an autoimmune disease. The autoimmune disease may be, for example, alopecia areata, ankylo sing spondylitis, antiphospholipid syndrome, Addison's disease, hemolytic anemia, warm autoimmune hemolytic anemia (wAIHA), anti-factor antibodies, heparin induced thrombocytopenia (HICT), sensitized transplant, autoimmune hepatitis, hepatitis, Behcet's disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Ménière's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, or Wegener's granulomatosis.

### EXAMPLES

The following materials and methods were used in the Examples set forth herein.

### Materials

Materials purchased from commercial vendors included monobasic sodium phosphate monohydrate (J.T. Baker), dibasic sodium phosphate anhydrous (J.T. Baker), succinic acid (TGI), sodium succinate (Macron), sodium chloride (J.T. Baker), citric acid monohydrate (AppliChem), hydrochloric acid (J.T. Baker), sodium hydroxide (Macron), high purity (low endotoxin) α-α-Trehalose dehydrate (Pfanstiehl), super-purified polysorbate 80-LQ (MH) (Croda).

The antibody used herein (comprising heavy chain SEQ ID NO:2 and light chain SEQ ID NO:1) has been formatted as an IgG1 G1m17allotype heavy chain, a fully lambda light chain lacking the terminal Lys (K446: EU Numbering), and with the Asn297Ala (EU Numbering) mutation that abolishes glycosylation at Asn297.

### Appearance Analysis

The appearance of all samples, including clarity, color, and visible particles, was examined against black and white background using a light box (Tianda Tianfa, Model YB-2).

### Measurement of pH

Sample pH was measured using a pH meter with an Inlab Micro electrode (Mettler Toledo, Model Seven Multi S40). The pH meter was calibrated prior to use each time with commercially available calibration solutions.

### Measurement of Protein Concentration

Protein concentration was determined by UV 280 nm readings using a NanoDrop 2000 spectrophotometer (Thermo Scientific). The extinction coefficient used in all studies was 1.447 AU ml mg⁻¹ cm⁻¹.

### Method for Osmolality Measurement

Osmolality was measured using an osmometer (Advanced Instruments, Advanced Multi-Sample Osmometer; Model Number 2020) without dilution of samples. Before and after testing, the testing accuracy of the osmometer was confirmed with clinical control 290 mOsm/kg reference solution.

### Differential Scanning Calorimetry

The capillary cell differential scanning calorimetry (DSC) was utilized to measure the thermal stability of proteins by detecting the difference in amount of heat required to increase the temperature of a sample and reference as a function of temperature. Specifically, DEC measures the thermal transition midpoint (Tm), which is an indicator of the relative stability of protein in solution. In brief, samples were diluted to about 1 mg ml⁻¹ with commercially available reference buffer. An aliquot of 400 μl of reference buffer was added into each odd-numbered well of a 96-well plate while an aliquot of 400 μl of each sample was added into the corresponding even-numbered well. The scanning temperature ranges from 10° C. to 100° C. with a scanning rate of 200° C. per hour. Data analysis was performed using MicroCal VP-Capillary DSC Automated data analysis software 2.0.

### Size Exclusion Chromatography

Size exclusion chromatography (SEC) was performed using an Agilent 1260 Infinity system with the TSKGel G3000SWXL size exclusion chromatography column (300×7.8 mm, 5 μM) at 25° C. Samples were diluted to 10 mg ml⁻¹ with mobile phase before SEC analysis and sample containing 100 μg protein was injected. An isocratic gradient was applied for 20 min at a flow rate of 1 ml min⁻¹. The mobile phase consisted of 50 mM sodium phosphate buffer 300 mM NaCl at pH 7.0±0.2. Data was collected by UV detector with detection wavelength set to 280 nm and data was analyzed using Waters Empower Software.

### Capillary Isoelectric Focusing

Capillary isoelectric focusing (cIEF) was performed to separate proteins based on charge differences in a pH gradient using Protein Simple iCE3 equipment with FC-coated cIEF cartridge. For monoclonal antibody (mAb) samples, 20 μg of each sample was mixed with 100 μL of master mix comprising isoelectric point (pI) marker 7.55/9.46, Servalyt 6-9, Servalyt 9-11, methyl cellulose solution. After mixing, the sample was focused for 1 min at 1500 V and 8 min at 3000 V. Detection wavelength was set to 280 nm and the charge variant distributions were evaluated in different pI ranges.

### Capillary Electrophoresis

Capillary electrophoresis (CE-SDS Caliper) was performed to separate dodecyl sulfate coated proteins based on size through a sieving polymer using a Beckman Coulter PA800 Enhanced or PA800 Plus instrument equipped with a photodiode array detector. For CE-SDS Caliper measured in reducing conditions samples were diluted to 4 mg ml⁻¹ by dilution solution (PB-CA), and then heated in the presence of 75 μl SDS sample buffer and 5 μl 2-mercaptoethanol at 70° C. for 10 min. For CE-SDS Caliper measured in non-reducing conditions samples were diluted to 4 mg ml⁻¹ by dilution solution (PB-CA), and then heated in the presence of 75 μl SDS sample buffer and 5 μl 100 mM NEM at 70° C. for 10 min Samples—prepared either in reducing or non-reducing conditions—were injected at the cathode with reverse polarity using −5 kV for 20 sec followed by separation at −15 kV and detection wavelength was set to 220 nm.

### Dynamic Light Scattering (DLS)

DLS is a technique which measures the degree to which light is scattered by a solution at a given temperature. The degree of scattering is proportional to the size (to the sixth power) and concentration (linear) of particle in solution. This technique is used to monitor submicron particles due to the profound effect of particle size on light scattering. The lowest pH (5.0) and highest pH (8.0) showed increases in size distribution. All other pH's showed no obvious differences.

### Example 1. Liquid Formulation Development Study to Determine how to Select Formulation Components—Buffer Species, pH, and Excipients—Impact on Stability of Liquid Formulations Comprising the Antibody

Select formulations of the antibody herein (comprising heavy chain SEQ ID NO:2 and light chain SEQ ID NO:1) present at 30 mg/ml with different concentrations of sodium phosphate, sodium succinate, NaCl, Trehalose, and PS-80 were prepared and the formulation properties—e.g. appearance, pH, protein concentration, osmolality, thermal stability, size purity, charge heterogeneity—were measured over time and compared.

Select formulations as detailed in Table 1 were prepared.

TABLE 1
Components and buffer conditions for select formulations.

| | | | | Concentration |
|---|---|---|---|---|
| Formu- | | Buffer | Tonicity Modifier, | of antibody |
| lation | pH | (25 mM) | Stabilizer, Surfactant | mg/mL |
| F1 | 7.5 | Sodium | 25 mM NaCl | 30 |
| | | Phosphate | | |
| F2 | 7.5 | Sodium | 25 mM NaCl + 90.5 mg/mL | 30 |
| | | Phosphate | Trehalose | |
| F3 | 7.5 | Sodium | 25 mM NaCl + 90.5 mg/mL | 30 |
| | | Phosphate | Trehalose + 0.01% w/v PS 80 | |
| F4 | 7.5 | Sodium | 150 mM NaCl | 30 |
| | | Phosphate | | |
| F5 | 7.5 | Sodium | 150 mM NaCl + 90.5 mg/mL | 30 |
| | | Phosphate | Trehalose | |
| F6 | 7.5 | Sodium | 150 mM NaCl + 90.5 mg/mL | 30 |
| | | Phosphate | Trehalose + 0.01% w/v PS 80 | |
| F7 | 7.0 | Sodium | 25 mM NaCl | 30 |
| | | Phosphate | | |
| F8 | 7.0 | Sodium | 25 mM NaCl + 90.5 mg/mL | 30 |
| | | Phosphate | Trehalose | |
| F9 | 7.0 | Sodium | 25 mM NaCl + 90.5 mg/mL | 30 |
| | | Phosphate | Trehalose + 0.01% w/v PS 80 | |
| F10 | 7.0 | Sodium | 150 mM NaCl | 30 |
| | | Phosphate | | |
| F11 | 7.0 | Sodium | 150 mM NaCl + 90.5 mg/mL | 30 |
| | | Phosphate | Trehalose | |
| F12 | 7.0 | Sodium | 150 mM NaCl + 90.5 mg/mL | 30 |
| | | Phosphate | Trehalose + 0.01% w/v PS 80 | |
| F13 | 6.5 | Sodium | 25 mM NaCl | 30 |
| | | Succinate | | |
| F14 | 6.5 | Sodium | 25 mM NaCl + 90.5 mg/mL | 30 |
| | | Succinate | Trehalose | |
| F15 | 6.5 | Sodium | 25 mM NaCl + 90.5 mg/mL | 30 |
| | | Succinate | Trehalose + 0.01% w/v PS 80 | |
| F16 | 6.5 | Sodium | 150 mM NaCl | 30 |
| | | Succinate | | |
| F17 | 6.5 | Sodium | 150 mM NaCl + 90.5 mg/mL | 30 |
| | | Succinate | Trehalose | |
| F18 | 6.5 | Sodium | 150 mM NaCl + 90.5 mg/mL | 30 |
| | | Succinate | Trehalose + 0.01% w/v PS 80 | |

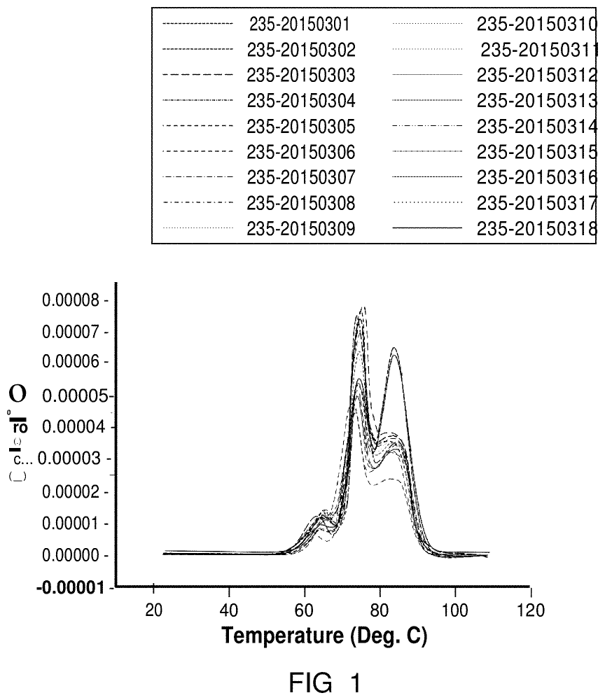

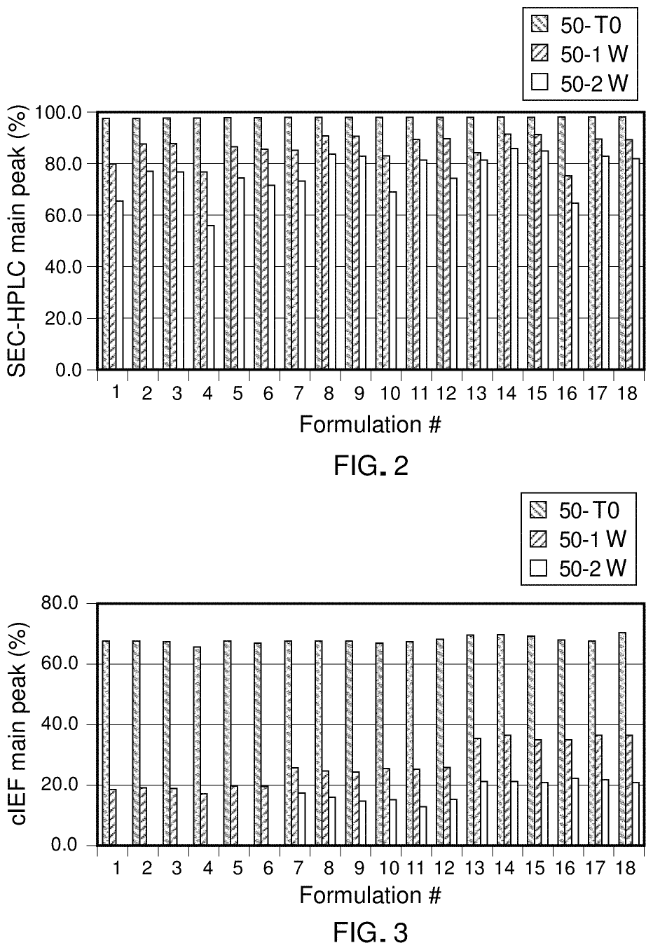

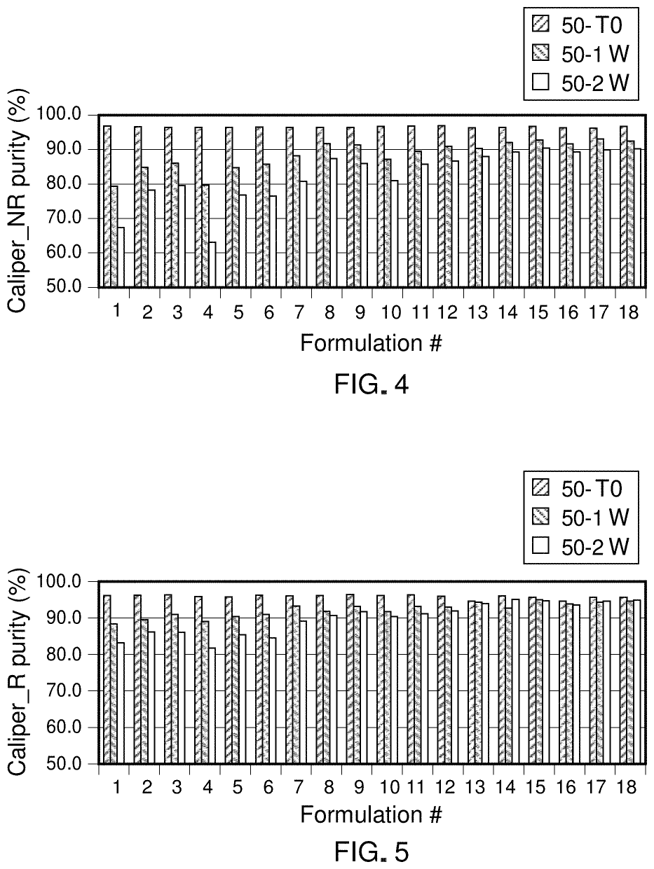

The formulations were monitored over time based on appearance, pH, protein concentration, osmolality, thermal stability, size purity and charge heterogeneity. All formulations tested exhibited no significant changes in pH, protein concentration, or osmolality for the duration of the study. In contrast, subsets of the formulations exhibited differences by appearance, thermal stability, size purity, and charge heterogeneity. Specifically, all formulations without Trehalose showed opalescence—an appearance seen in highly dispersed systems with little opacity—after 14 days at accelerated conditions (50° C.). All other formulations remained colorless, clear, and free of visible particles after 4 weeks at 2-8° C. and 14 days at accelerated conditions (50° C.). These results indicate that the Trehalose component confers stability to the liquid formulations comprising M281. The thermal stability of the different formulations was determined by differential scanning calorimetry (FIG. 1). The results indicate that increased stability is conferred to formulations containing either lower sodium chloride concentration or containing Trehalose. The size purity of the different formulations was determined by size exclusion chromatography (FIG. 2). The results indicate that formulations containing low sodium chloride concentration and Trehalose at pH 6.5 exhibit the highest size purity stability over time. The charge heterogeneity of the different formulations was determined by capillary isoelectric focusing (cIEF) at accelerated conditions (50° C.) (FIG. 3). Varying the concentration of different stabilizers—e.g. sodium chloride, Trehalose, PS 80—did not significantly affect the charge heterogeneity of formulations over time. In contrast, pH of the formulations has a significant effect, specifically formulations at the pH 6.5 exhibited better maintenance of charge heterogeneity after 1 and 2 weeks compared to formulations at pH 7 or pH 7.5. The purity of the different formulations was measured by CE-SDS Caliper in non-reduced accelerated conditions (FIG. 4) and in reduced accelerated conditions (50° C.) (FIG. 5). The results indicate that increased stability over time is conferred to formulations containing Trehalose and buffered at pH 6.5 compared to pH 7 or pH 7.5.

Overall, formulations with Trehalose were more stable that those without Trehalose. Formulations with 25 mM NaCl were more stable than those containing 150 mM NaCl. In conclusion, results of this formulation screen indicated that, among the tested formulations, the formulation with 25 mM NaCl and 90.5 mg/mL Trehalose exhibits the highest stability and the stability is sufficiently maintained over 1 and 2 weeks.

To determine how formulation pH affects formulation stability, select formulations of the antibody (10 mg/ml) in 25 mM citric and dibasic phosphate buffer were prepared at different pH and the formulation properties (e.g. appearance, pH, protein concentration, osmolality, thermal stability, size purity, and charge heterogeneity, etc.) were measured and compared. Select formulations as detailed in Table 2 were prepared.

TABLE 2
Buffer conditions for select formulations

| Formu- | | | Antibody |
|---|---|---|---|
| lation | pH | Buffer (25 mM) | mg/mL |
| F19 | 5.0 | citric acid & dibasic phosphate buffer | 10 |
| F20 | 5.5 | citric acid & dibasic phosphate buffer | 10 |
| F21 | 6.0 | citric acid & dibasic phosphate buffer | 10 |
| F22 | 6.5 | citric acid & dibasic phosphate buffer | 10 |
| F23 | 7.0 | citric acid & dibasic phosphate buffer | 10 |
| F24 | 7.5 | citric acid & dibasic phosphate buffer | 10 |
| F25 | 8.0 | citric acid & dibasic phosphate buffer | 10 |

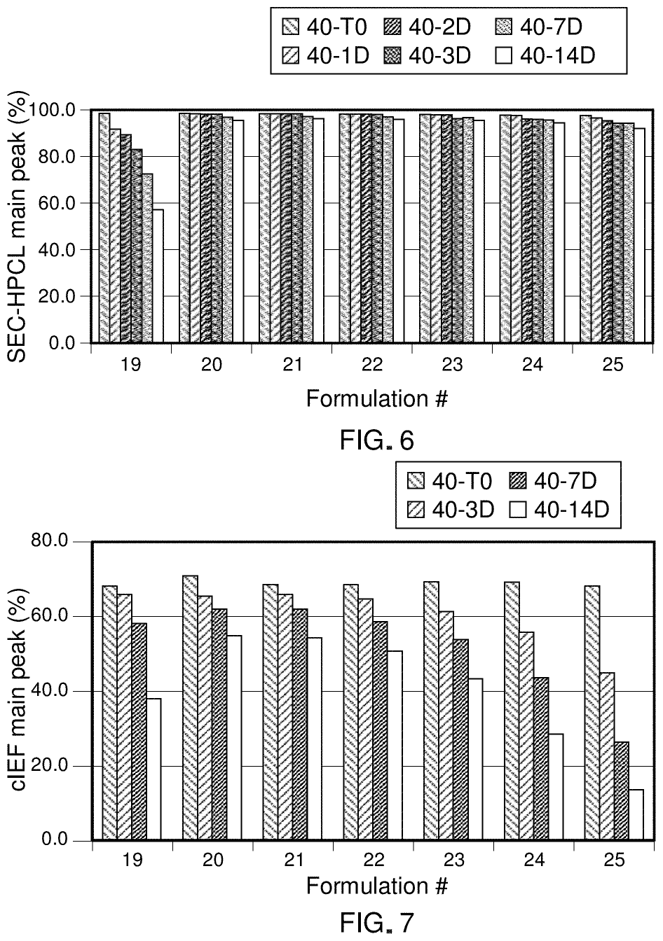

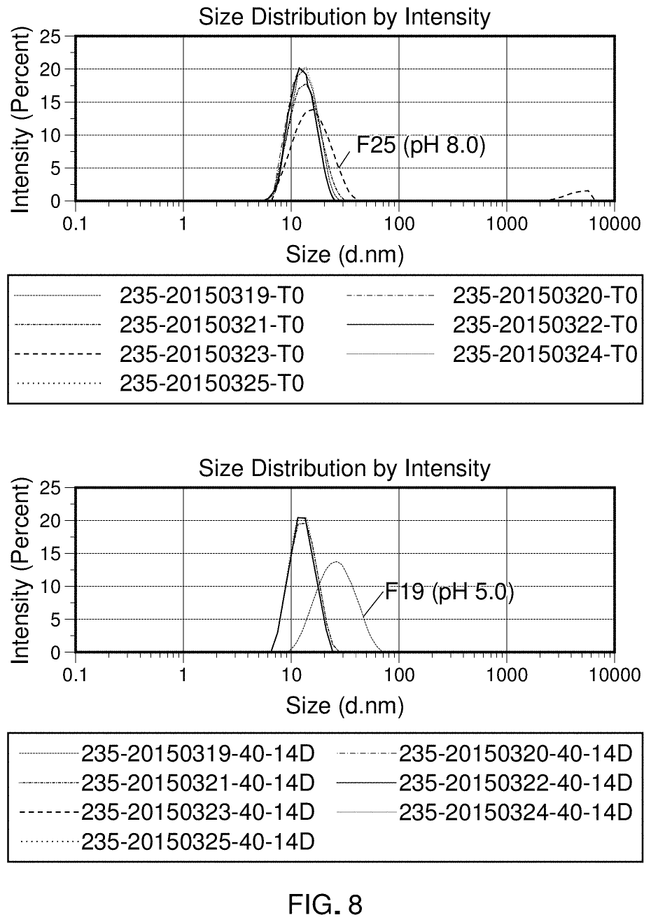

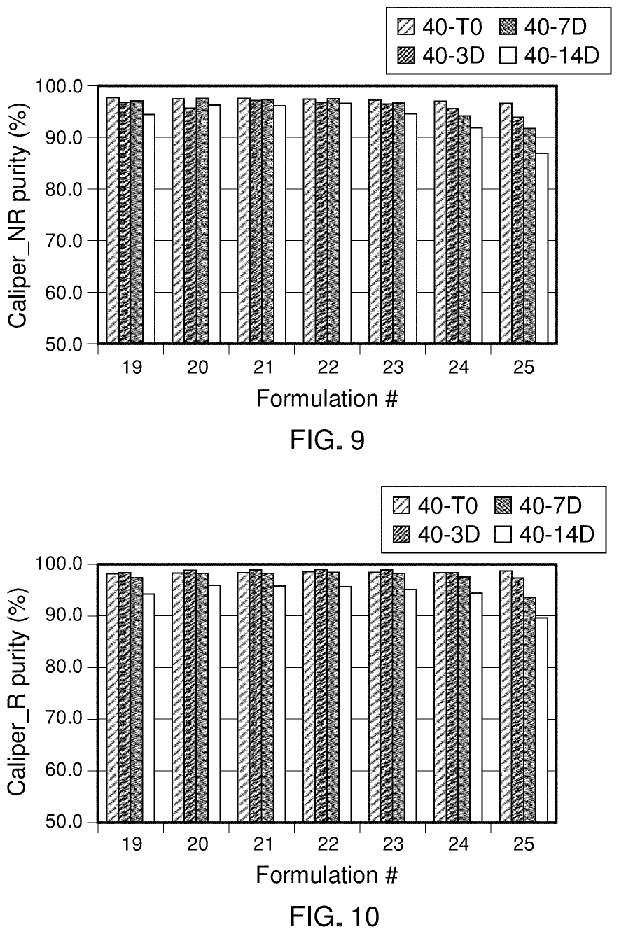

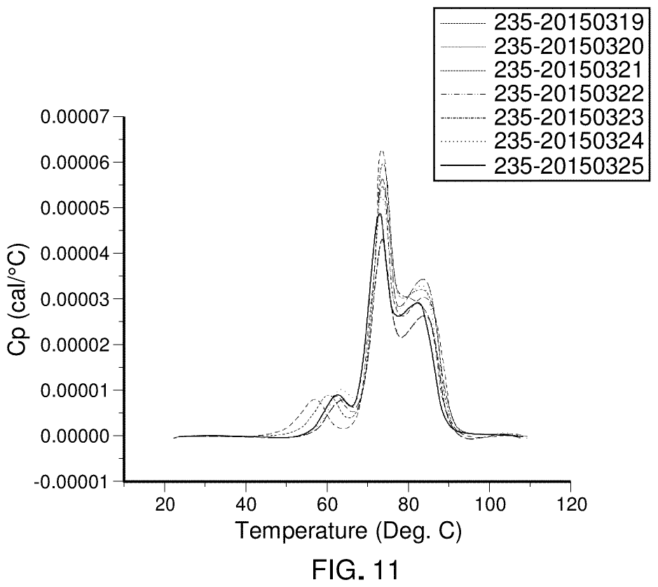

The formulations were monitored over time based on appearance, pH, protein concentration, osmolality, thermal stability, size purity and charge heterogeneity. All formulations exhibited no significant changes in appearance, pH, protein concentration, or osmolality for the duration of the study. In contrast, subsets of the formulations exhibited differences by size purity, charge heterogeneity, and thermal stability. The size purity of the different formulations was determined by size exclusion chromatography (FIG. 6). Formulations at pH 5, pH 7, pH 7.5, and pH-8 exhibited decreased amounts of the target sized molecules (referred to as main or target peak) compared to formulations at pH 6 or 6.5. These results indicate that formulations at pH 6 and 6.5 exhibit higher size purity. The charge heterogeneity of the different formulations was determined by capillary isoelectric focusing (cIEF) at accelerated conditions (50° C.) (FIG. 7). Formulations at pH 5.5, 6.0 and 6.5 exhibited better maintenance of charge heterogeneity compared to formulations buffered at the higher pHs tested. The size distribution of the different formulations was determined by dynamic light scattering (FIG. 8). The formulations at pH 5.5-7.5 showed no significant changes in size distribution. In contrast, formulations at pH 5 and pH 8 showed changes in size distribution, indicating that formulations at pH 5 or 8 are not stable and may form degradation products over time. The purity of the different formulations was measured by CE-SDS Caliper in non-reduced accelerated conditions (50° C.) (FIG. 9) and in reduced accelerated conditions (50° C.) (FIG. 10). The results indicate that increased stability is conferred to formulations buffered at pH 5, 5.5, 6, or 6.5 and this stability is preserved in presence of reducing (in presence of β-mercaptoethanol) or non-reducing (in presence of N-ethylmaleimide) accelerated conditions. The thermal stability of the different formulations was determined by differential scanning calorimetry (FIG. 11). The formulations buffered at pH 5.5, pH 6, and pH 6.5 exhibited higher thermal stability compared to those buffered at pH 7, pH 7.5, or pH 8. The highest thermal stability was conferred to formulations buffered at pH 6.5 given that Formulation 22 had the highest Tm onset and Tm1 values.

In conclusion, the results of this liquid formulation development study indicate formulation stability was conferred to liquid formulations of the antibody prepared with 25 mM NaCl and 90.5 mg/mL Trehalose and buffered at pH 6.5.

### Example 2. Stability Analysis Study to Determine Stability of Select Formulations when Exposed to Mechanical, Chemical, and Thermal Stresses

To examine and compare the stability of select formulations in the presence of stresses, select formulations were prepared and exposed to different stresses, including mechanical agitation, visible light, UV light, high temperature, multiple freeze-thaw, and oxidizing agents.

### Results

Select formulations as detailed in Table 3 were prepared.

TABLE 3
Components and buffer conditions for select formulations

| Formu- | | Buffer | Tonicity Modifier, | Antibody |
|---|---|---|---|---|
| lation | pH | (25 mM) | Stabilizer, Surfactant | mg/mL |
| F26 | 6.5 | Sodium | 25 mM NaCl + 90.5 mg/mL | 10 |
| | | Phosphate | Trehalose + 0.01% w/v PS 80 | |
| F27 | 6.5 | Sodium | 25 mM NaCl + 90.5 mg/mL | 10 |
| | | Succinate | Trehalose + 0.01% w/v PS 80 | |
| F28 | 6.5 | Sodium | 25 mM NaCl + 90.5 mg/mL | 30 |
| | | Phosphate | Trehalose + 0.01% w/v PS 80 | |
| F29 | 6.5 | Sodium | 25 mM NaCl + 90.5 mg/mL | 30 |
| | | Succinate | Trehalose + 0.01% w/v PS 80 | |
| F30 | 6.5 | Sodium | 25 mM NaCl + 0.01% w/v PS 80 | 30 |
| | | Phosphate | | |
| F31 | 6.5 | Sodium | 25 mM NaCl + 0.01% w/v PS 80 | 30 |
| | | Succinate | | |

### Agitation Stress

The formulations were exposed to mechanical agitation at 250 rpm at 25° C. for 5 or 10 days. The formulations exhibited no significant change in appearance, protein concentration, pI, size purity, or charge purity. Notably, the formulations exhibited similar proportion of main peak, acid peak, and basic peak in the clEF assay, size purity measured by the non-reduced and reduced Caliper assay, and average particle size and PdI of DLS assay compared to each other and over time (Table 4). In SEC assay, agitation led to a slight decline on main peak percentage relative to total content and increased content of aggregates.

TABLE 4
Formulation components and buffer conditions

| | | | | | Agitation study | |
|---|---|---|---|---|---|---|
| Assays | | No. | Sample ID | T0 | 5 D | 10 D |
| Appearance | | F26 | 20150401 | colorless, | colorless, | colorless, |
| | | F27 | 20150402 | clear, and | clear, and | clear, and |
| | | | | free of | free of | free of |
| | | | | visible | visible | visible |
| | | | | particle | particle | particle |
| Conc., mg/mL | | F26 | 20150401 | 10.7 | 10.5 | 10.5 |
| | | F27 | 20150402 | 10.0 | 9.7 | 9.7 |
| pH | | F26 | 20150401 | 6.42 | 6.41 | 6.38 |
| | | F27 | 20150402 | 6.55 | 6.52 | 6.54 |
| SEC | Main | F26 | 20150401 | 98.0 | 97.9 | 97.3 |
| | peak % | F27 | 20150402 | 98.0 | 97.7 | 97.2 |
| | HMW | F26 | 20150401 | 2.0 | 2.1 | 2.7 |
| | peak % | F27 | 20150402 | 2.0 | 2.2 | 2.8 |
| | LMW | F26 | 20150401 | ND | ND | ND |
| | peak % | F27 | 20150402 | ND | ND | 0.1 |
| cIEF | pI | F26 | 20150401 | 8.81 | NA | 8.82 |
| | | F27 | 20150402 | 8.82 | NA | 8.81 |
| | Main | F26 | 20150401 | 65.9 | NA | 64.2 |
| | peak % | F27 | 20150402 | 64.6 | NA | 65.0 |
| | Acid | F26 | 20150401 | 31.7 | NA | 32.6 |
| | peak % | F27 | 20150402 | 32.3 | NA | 32.1 |
| | Basic | F26 | 20150401 | 2.4 | NA | 3.2 |
| | peak % | F27 | 20150402 | 3.1 | NA | 2.9 |
| Reduced | Purity % | F26 | 20150401 | 97.7 | NA | 98.3 |
| Caliper | | F27 | 20150402 | 97.5 | NA | 97.4 |
| | LC | F26 | 20150401 | 43.9 | NA | 42.3 |
| | Size, kDa | F27 | 20150402 | 43.6 | NA | 41.8 |
| | HC | F26 | 20150401 | 56.8 | NA | 56.0 |
| | Size, kDa | F27 | 20150402 | 56.8 | NA | 55.1 |
| Non- | Purity % | F26 | 20150401 | 98.0 | NA | 98.3 |
| reduced | | F27 | 20150402 | 98.0 | NA | 98.4 |
| Caliper | Size, kDa | F26 | 20150401 | 169.5 | NA | 162.7 |
| | | F27 | 20150402 | 170.8 | NA | 160.9 |
| DLS | Z-Ave | F26 | 20150401 | 15.5 | NA | 15.4 |
| | (d.nm) | F27 | 20150402 | 15.5 | NA | 15.4 |
| | PdI | F26 | 20150401 | 0.10 | NA | 0.09 |
| | | F27 | 20150402 | 0.11 | NA | 0.11 |

### Thermal Stress

The formulations were exposed to thermal Stress at 40° C. for 5 or 10 days. The formulations exhibited no significant change in appearance, protein concentration, pI, size purity as assessed by SEC or DLS, or charge purity (Table 5). The formulations exhibited decline in main peak of clEF assay and an increase in percentage of acid specific peaks, however all formulations tested (Table 3) exhibited similar magnitude of changes.

TABLE 5
Results of SEC, cIEF, reduced Caliper, Non-reduced Caliper, appearance, protein
concentration, pH for Formulation 26 and 27 after exposure to thermal stress

| | | | | | Thermo stability study | |
|---|---|---|---|---|---|---|
| Assays | | No. | Sample ID | T0 | 5 D | 10 D |
| Appearance | | F26 | 20150401 | colorless, | colorless, | colorless, |
| | | F27 | 20150402 | clear, and | clear, and | clear, and |
| | | | | free of | free of | free of |
| | | | | visible | visible | visible |
| | | | | particle | particle | particle |
| Conc., mg/mL | | F26 | 20150401 | 10.7 | 10.5 | 10.5 |
| | | F27 | 20150402 | 10.0 | 9.7 | 9.7 |
| pH | | F26 | 20150401 | 6.42 | 6.40 | 6.41 |
| | | F27 | 20150402 | 6.55 | 6.51 | 6.53 |
| SEC | Main | F26 | 20150401 | 98.0 | 98.1 | 97.8 |
| | peak % | F27 | 20150402 | 98.0 | 98.0 | 97.9 |
| | HMW | F26 | 20150401 | 2.0 | 1.7 | 1.9 |
| | peak % | F27 | 20150402 | 2.0 | 1.9 | 1.9 |
| | LMW | F26 | 20150401 | ND | 0.1 | 0.3 |
| | peak % | F27 | 20150402 | ND | 0.1 | 0.2 |
| cIEF | pI | F26 | 20150401 | 8.81 | NA | 8.82 |
| | | F27 | 20150402 | 8.82 | NA | 8.81 |
| | Main | F26 | 20150401 | 65.9 | NA | 54.3 |
| | peak % | F27 | 20150402 | 64.6 | NA | 56.3 |
| | Acid | F26 | 20150401 | 31.7 | NA | 41.2 |
| | peak % | F27 | 20150402 | 32.3 | NA | 39.9 |
| | Basic | F26 | 20150401 | 2.4 | NA | 4.4 |
| | peak % | F27 | 20150402 | 3.1 | NA | 3.8 |
| Reduced | Purity % | F26 | 20150401 | 97.7 | NA | 97.3 |
| Caliper | | F27 | 20150402 | 97.5 | NA | 97.4 |
| | LC | F26 | 20150401 | 43.9 | NA | 42.0 |
| | Size, kDa | F27 | 20150402 | 43.6 | NA | 42.8 |
| | HC | F26 | 20150401 | 56.8 | NA | 55.5 |
| | Size, kDa | F27 | 20150402 | 56.8 | NA | 56.6 |
| Non- | Purity % | F26 | 20150401 | 98.0 | NA | 97.9 |
| reduced | | F27 | 20150402 | 98.0 | NA | 98.1 |
| Caliper | Size, kDa | F26 | 20150401 | 169.5 | NA | 160.1 |
| | | F27 | 20150402 | 170.8 | NA | 163.5 |
| DLS | Z-Ave | F26 | 20150401 | 15.5 | NA | 15.6 |
| | (d.nm) | F27 | 20150402 | 15.5 | NA | 15.3 |
| | PdI | F26 | 20150401 | 0.10 | NA | 0.10 |
| | | F27 | 20150402 | 0.11 | NA | 0.10 |

### Visible Light Stress

The formulations were exposed to visible light stress of 5000 lux at 25° C. for 5 or10 days. The formulations did not exhibit significantly different appearance, protein concentration, pH, pI, non-reduced Caliper purity, average particle size, or PdI by DLS assay (Table 6). Slight decreases in protein purity by SEC, cIEF and reduced Caliper were observed.

TABLE 6
Results of SEC, cIEF, reduced Caliper, Non-reduced Caliper, appearance, protein concentration,
pH for Formulation 26 and 27 after exposure to visible light stress

| | | | | | Visible light sensitivity study | | | |
|---|---|---|---|---|---|---|---|---|
| | | | | | Visible light | | Protected from light | |
| Assays | | No. | Sample ID | T0 | 5 D | 10 D | 5 D | 10 D |
| Appearance | | F26 | 20150401 | colorless, | colorless, | colorless, | colorless, | colorless, |
| | | F27 | 20150402 | clear, and | clear, and | clear, and | clear, and | clear, and |
| | | | | free of | free of | free of | free of | free of |
| | | | | visible | visible | visible | visible | visible |
| | | | | particle | particle | particle | particle | particle |
| Conc., mg/mL | | F26 | 20150401 | 10.5 | 10.6 | 10.6 | 10.5 | 10.5 |
| | | F27 | 20150402 | 9.7 | 9.7 | 9.7 | 9.8 | 9.7 |
| pH | | F26 | 20150401 | 6.39 | 6.40 | 6.39 | 6.40 | 6.39 |
| | | F27 | 20150402 | 6.51 | 6.53 | 6.51 | 6.52 | 6.51 |
| SEC | Main | F26 | 20150401 | 98.0 | 97.3 | 96.3 | 98.2 | 98.2 |
| | peak % | F27 | 20150402 | 98.0 | 96.8 | 96.0 | 98.2 | 98.2 |
| | HMW | F26 | 20150401 | 2.0 | 2.6 | 3.5 | 1.8 | 1.8 |
| | peak % | F27 | 20150402 | 2.0 | 3.1 | 3.8 | 1.8 | 1.8 |
| | LMW | F26 | 20150401 | ND | 0.1 | 0.3 | ND | 0.1 |
| | peak % | F27 | 20150402 | ND | 0.1 | 0.2 | ND | ND |
| cIEF | pI | F26 | 20150401 | 8.81 | NA | 8.82 | NA | 8.82 |
| | | F27 | 20150402 | 8.82 | NA | 8.81 | NA | 8.81 |
| | Main | F26 | 20150401 | 65.9 | NA | 59.7 | NA | 64.5 |
| | peak % | F27 | 20150402 | 64.6 | NA | 59.4 | NA | 64.5 |
| | Acid | F26 | 20150401 | 31.7 | NA | 37.2 | NA | 33.0 |
| | peak % | F27 | 20150402 | 32.3 | NA | 37.3 | NA | 32.3 |
| | Basic | F26 | 20150401 | 2.4 | NA | 3.1 | NA | 2.5 |
| | peak % | F27 | 20150402 | 3.1 | NA | 3.3 | NA | 3.2 |
| Reduced | Purity % | F26 | 20150401 | 97.7 | NA | 96.3 | NA | 98.4 |
| Caliper | | F27 | 20150402 | 97. [symbol] | NA | 95.2 | NA | 95.8 |
| | LC | F26 | 20150401 | 43.9 | NA | 41.7 | NA | 41.5 |
| | Size, kDa | F27 | 20150402 | 43.6 | NA | 41.9 | NA | 42.4 |
| | HC | F26 | 20150401 | 56.8 | NA | 55.5 | NA | 55.1 |
| | Size, kDa | F27 | 20150402 | 56.8 | NA | 55.7 | NA | 56.3 |
| Non- | Purity % | F26 | 20150401 | 98.0 | NA | 97.7 | NA | 98.3 |
| reduced | | F27 | 20150402 | 98.0 | NA | 97.9 | NA | 98.2 |
| Caliper | Size, kDa | F26 | 20150401 | 169.5 | NA | 163.9 | NA | 162.9 |
| | | F27 | 20150402 | 170.8 | NA | 160.9 | NA | 160.8 |
| DLS | Z-Ave | F26 | 20150401 | 15.5 | NA | 16.2 | NA | 15.4 |
| | (d.nm) | F27 | 20150402 | 15.5 | NA | 15.3 | NA | 15.2 |
| | PdI | F26 | 20150401 | 0.10 | NA | 0.13 | NA | 0.07 |
| | | F27 | 20150402 | 0.11 | NA | 0.09 | NA | 0.08 |

[symbol]

indicates data missing or illegible when filed

### UV-Light Stress

The formulations were exposed to UV-light stress of 200 w/m² at 25° C. for 10 hours. The formulations did not exhibit significantly different appearance, protein concentration, pH, pI, non-reduced Caliper purity, and average particle size or PdI by DLS assay (Table 7). Decreases in protein purity by SEC, cIEF and reduced Caliper were observed.

TABLE 7
Results of SEC, cIEF, reduced Caliper, Non-reduced Caliper, appearance, protein
concentration, pH for Formulation 26 and 27 after exposure to UV-light stress

| | | | | | UV light sensitivity study | |
|---|---|---|---|---|---|---|
| | | | | | UV | protected from |
| Assays | | No. | Sample ID | T0 | light, 10 h | light, 10 h |
| Appearance | | F26 | 20150401 | colorless, | colorless, | colorless, |
| | | F27 | 20150402 | clear, and | clear, and | clear, and |
| | | | | free of | free of | free of |
| | | | | visible | visible | visible |
| | | | | particle | particle | particle |
| Conc., mg/mL | | F26 | 20150401 | 10.7 | 10.6 | 10.5 |
| | | F27 | 20150402 | 10.0 | 9.8 | 9.7 |
| pH | | F26 | 20150401 | 6.42 | 6.39 | 6.39 |
| | | F27 | 20150402 | 6.55 | 6.51 | 6.51 |
| SEC | Main | F26 | 20150401 | 98.0 | 96.7 | 98.2 |
| | peak % | F27 | 20150402 | 98.0 | 96.2 | 98.2 |
| | HMW | F26 | 20150401 | 2.0 | 3.2 | 1. [symbol] |
| | peak % | F27 | 20150402 | 2.0 | 3.8 | 1. [symbol] |
| | LMW | F26 | 20150401 | ND | 0.1 | ND |
| | peak % | F27 | 20150402 | ND | 0.1 | ND |
| cIEF | pI | F26 | 20150401 | 8.81 | 8.81 | 8.81 |
| | | F27 | 20150402 | 8.82 | 8.81 | 8.81 |
| | Main | F26 | 20150401 | 65.9 | 62.0 | 66.8 |
| | peak % | F27 | 20150402 | 64.6 | 61.7 | 65.5 |
| | Acid | F26 | 20150401 | 31.7 | 35.3 | 30.4 |
| | peak % | F27 | 20150402 | 32.3 | 35.3 | 31.5 |
| | Basic | F26 | 20150401 | 2.4 | 2.7 | 2.8 |
| | peak % | F27 | 20150402 | 3.1 | 3.0 | 3.1 |
| Reduced | Purity % | F26 | 20150401 | 97.7 | 96.5 | 97.8 |
| Caliper | | F27 | 20150402 | 97.5 | 96.5 | 97.7 |
| | LC | F26 | 20150401 | 43.9 | 43.3 | 43.1 |
| | Size, kDa | F27 | 20150402 | 43.6 | 42.7 | 42.8 |
| | HC | F26 | 20150401 | 56.8 | 56.4 | 56.0 |
| | Size, kDa | F27 | 20150402 | 56.8 | 55.5 | 55.7 |
| Non- | Purity % | F26 | 20150401 | 98.0 | 98.2 | 98.3 |
| reduced | | F27 | 20150402 | 98.0 | 98.2 | 98.4 |
| Caliper | Size, kDa | F26 | 20150401 | 169.5 | 162.1 | 161.6 |
| | | F27 | 20150402 | 170.8 | 163.7 | 164.5 |
| DLS | Z-Ave | F26 | 20150401 | 15.5 | 15.2 | 15.6 |
| | (d.nm) | F27 | 20150402 | 15.5 | 15.2 | 15.3 |
| | PdI | F26 | 20150401 | 0.10 | 0.08 | 0.10 |
| | | F27 | 20150402 | 0.11 | 0.09 | 0.08 |

[symbol]

indicates data missing or illegible when filed

### Oxidation Stress

The formulations were exposed oxidation Stress with exposure to 1% H₂O₂ at 2-8° C. for 6 hours. The formulations did not exhibit significantly different appearance, protein concentration, pH, pI, SEC purity, reduced and non-reduced Caliper purity, or average particle size and PdI by DLS assay (Table 8). Slight decreases were observed for cIEF.

TABLE 8
Results of SEC, cIEF, reduced Caliper, Non-reduced Caliper, appearance, protein
concentration, pH for Formulation 26 and 27 after exposure to oxidation stress

| | | | | | oxidation | | |
|---|---|---|---|---|---|---|---|
| Assays | | No. | Sample ID | T0 | 1 h | 3 h | 6 h |
| Appearance | | F26 | 20150401 | colorless, | colorless, | colorless, | colorless, |
| | | F27 | 20150402 | clear, and | clear, and | clear, and | clear, and |
| | | | | free of | free of | free of | free of |
| | | | | visible | visible | visible | visible |
| | | | | particle | particle | particle | particle |
| Conc., mg/mL | | F26 | 20150401 | 10.5 | 11.2 | 11.3 | 11.3 |
| | | F27 | 20150402 | 9.8 | 10.5 | 10.6 | 10.5 |
| pH | | F26 | 20150401 | 6.39 | 6.28 | 6.30 | 6.31 |
| | | F27 | 20150402 | 6.51 | 6.42 | 6.42 | 6.43 |
| SEC | Main | F26 | 20150401 | 9 [symbol] .1 | 98.3 | 98.3 | 98.4 |
| | peak % | F27 | 20150402 | 9 [symbol] .1 | 98.2 | 98.2 | 98.2 |
| | HMW | F26 | 20150401 | 1.9 | 1.6 | 1.6 | 1.5 |
| | peak % | F27 | 20150402 | 1.9 | 1.7 | 1.6 | 1.6 |
| | LMW | F26 | 20150401 | ND | 0.1 | 0.1 | 0.2 |
| | peak % | F27 | 20150402 | ND | 0.1 | 0.1 | 0.2 |
| cIEF | pI | F26 | 20150401 | 8.82 | NA | NA | 8.81 |
| | | F27 | 20150402 | 8.82 | NA | NA | 8.81 |
| | Main | F26 | 20150401 | 65.6 | NA | NA | 62.7 |
| | peak % | F27 | 20150402 | 64.4 | NA | NA | 61.4 |
| | Acid | F26 | 20150401 | 31.9 | NA | NA | 34.4 |
| | peak % | F27 | 20150402 | 32.8 | NA | NA | 35.7 |
| | Basic | F26 | 20150401 | 2.4 | NA | NA | 2.9 |
| | peak % | F27 | 20150402 | 2.7 | NA | NA | 2.9 |
| Reduced | Purity % | F26 | 20150401 | 97.6 | NA | NA | 97.5 |
| Caliper | | F27 | 20150402 | 97.6 | NA | NA | 97.3 |
| | LC | F26 | 20150401 | 43.9 | NA | NA | 43.6 |
| | Size, kDa | F27 | 20150402 | 43.7 | NA | NA | 43.4 |
| | HC | F26 | 20150401 | 57.0 | NA | NA | 58.1 |
| | Size, kDa | F27 | 20150402 | 56.8 | NA | NA | 57.8 |
| Non- | Purity % | F26 | 20150401 | 97.9 | NA | NA | 97.9 |
| reduced | | F27 | 20150402 | 98.1 | NA | NA | 97.8 |
| Caliper | Size, kDa | F26 | 20150401 | 167.1 | NA | NA | 167.2 |
| | | F27 | 20150402 | 166.0 | NA | NA | 167.2 |
| DLS | Z-Ave | F26 | 20150401 | 15.1 | 15.8 | 14.7 | 14.9 |
| | (d.nm) | F27 | 20150402 | 15.8 | 14.9 | 14.8 | 14.7 |
| | PdI | F26 | 20150401 | 0.09 | 0.15 | 0.09 | 0.09 |
| | | F27 | 20150402 | 0.13 | 0.09 | 0.09 | 0.08 |

[symbol]

indicates data missing or illegible when filed

### Freeze-Thaw Stress

The formulations were exposed to freeze-thaw from −80° C. to room temperature (RT) for up to 10 cycles. The formulations did not exhibit significantly different appearance, protein concentration, SEC purity, pI, the proportion of main peak, acid peak, and basic peak of clEF assay, purity of non-reduced and reduced Caliper assay, or average particle size and PdI of DLS assay (Table 9).

TABLE 9
Results of SEC, cIEF, reduced Caliper, Non-reduced Caliper, appearance, protein
concentration, pH for Formulation 26 and 27 after exposure to oxidation stress

| Assays | | No. | Sample ID | T0 | Freeze-thaw | | |
|---|---|---|---|---|---|---|---|
| Appearance | | F28 | 20150401-FT | colorless, | colorless, | colorless, | colorless, |
| | | F29 | 20150402-FT | clear, and | clear, and | clear, and | clear, and |
| | | F30 | 20150403-FT | free of | free of | free of | free of |
| | | F31 | 20150404-FT | visible | visible | visible | visible |
| | | | | particle | particle | particle | particle |
| Conc., mg/mL | | F28 | 20150401-FT | 33.9 | 38.0 | 38.5 | 39.1 |
| | | F29 | 20150402-FT | 34.4 | 37.5 | 41.2 | 3 [symbol] |
| | | F30 | 20150403-FT | 29.0 | 34.0 | 30.4 | 32.1 |
| | | F31 | 20150404-FT | 29.2 | 35.1 | 29.5 | 29.5 |
| pH | | F28 | 20150401-FT | 6.40 | 6.38 | 6.35 | 6.37 |
| | | F29 | 20150402-FT | 5.48 | 6.48 | 6.45 | 6.46 |
| | | F30 | 20150403-FT | 6.56 | 6.54 | 6.53 | 6.52 |
| | | F31 | 20150404-FT | 6.53 | 6.51 | 6.51 | 6.51 |
| Osmolality, | | F28 | 20150401-FT | 482 | NA | NA | NA |
| mOsm/kg | | F29 | 20150402-FT | 479 | NA | NA | NA |
| | | F30 | 20150403-FT | 113 | NA | NA | NA |
| | | F31 | 20150404-FT | 99 | NA | NA | NA |
| SEC | Main | F28 | 20150401-FT | 97.7 | 97.7 | 97.4 | 97.3 |
| | peak % | F29 | 20150402-FT | 97. [symbol] | 97.6 | 97.3 | 97.3 |
| | | F30 | 20150403-FT | 97. [symbol] | 97.5 | 97.1 | 97.0 |
| | | F31 | 20150404-FT | 97. [symbol] | 97.8 | 97.2 | 97.1 |
| | HMW | F28 | 20150401-FT | 2.3 | 2.3 | 2. [symbol] | 2.6 |
| | peak % | F29 | 20150402-FT | 2.4 | 2.4 | 2. [symbol] | 2.7 |
| | | F30 | 20150403-FT | 2.4 | 2.5 | 2.8 | 2.9 |
| | | F31 | 20150404-FT | 2.4 | 2.4 | 2.8 | 2. [symbol] |
| | LMW | F28 | 20150401-FT | ND | ND | ND | 0.1 |
| | peak % | F29 | 20150402-FT | ND | ND | ND | ND |
| | | F30 | 20150403-FT | ND | ND | ND | ND |
| | | F31 | 20150404-FT | ND | ND | 0.1 | ND |
| cIEF | pI | F28 | 20150401-FT | 8.83 | NA | NA | 8.80 |
| | | F29 | 20150402-FT | 8.83 | NA | NA | 8.79 |
| | | F30 | 20150403-FT | 8. [symbol] 2 | NA | NA | 8.79 |
| | | F31 | 20150404-FT | 8. [symbol] 2 | NA | NA | 8.79 |
| | Main | F28 | 20150401-FT | 65.7 | NA | NA | 86.1 |
| | peak % | F29 | 20150402-FT | 65.4 | NA | NA | 86.2 |
| | | F30 | 20150403-FT | 64.2 | NA | NA | [symbol] |
| | | F31 | 20150404-FT | 64.9 | NA | NA | [symbol] |
| | Acid | F28 | 20150401-FT | 31.7 | NA | NA | 30.9 |
| | peak % | F29 | 20150402-FT | 31.8 | NA | NA | 30.7 |
| | | F30 | 20150403-FT | 32.8 | NA | NA | 31.1 |
| | | F31 | 20150404-FT | 32.3 | NA | NA | 30.5 |
| | Basic | F28 | 20150401-FT | 2.7 | NA | NA | 3.0 |
| | peak % | F29 | 20150402-FT | 2. [symbol] | NA | NA | 3.1 |
| | | F30 | 20150403-FT | 3.0 | NA | NA | 3.1 |
| | | F31 | 20150404-FT | 2.9 | NA | NA | 3.2 |
| Reduced | Purity % | F28 | 20150401-FT | 97.7 | NA | NA | 98.3 |
| Caliper | | F29 | 20150402-FT | 97.5 | NA | NA | 97.8 |
| | | F30 | 20150403-FT | 97.5 | NA | NA | 97.8 |
| | | F31 | 20150404-FT | 97.6 | NA | NA | 98.4 |
| | LC | F28 | 20150401-FT | 43.5 | NA | NA | 42.9 |
| | Size, kDa | F29 | 20150402-FT | 43.7 | NA | NA | 43.4 |
| | | F30 | 20150403-FT | 43.7 | NA | NA | 43.3 |
| | | F31 | 20150404-FT | 43.8 | NA | NA | 42.9 |
| | HC | F28 | 20150401-FT | 5 [symbol] | NA | NA | 5 [symbol] |
| | Size, kDa | F29 | 20150402-FT | 5 [symbol] | NA | NA | 57.1 |
| | | F30 | 20150403-FT | 57.0 | NA | NA | 57.3 |
| | | F31 | 20150404-FT | 57.0 | NA | NA | [symbol] |
| Non- | Purity % | F28 | 20150401-FT | 97.9 | NA | NA | 98.2 |
| reduced | | F29 | 20150402-FT | 97.9 | NA | NA | 98.3 |
| Caliper | | F30 | 20150403-FT | 97.7 | NA | NA | 98.1 |
| | | F31 | 20150404-FT | 97.7 | NA | NA | 98.1 |
| | Size, kDa | F28 | 20150401-FT | 170.1 | NA | NA | 162.0 |
| | | F29 | 20150402-FT | 169.7 | NA | NA | 161.4 |
| | | F30 | 20150403-FT | 169.7 | NA | NA | 162.5 |
| | | F31 | 20150404-FT | 169.9 | NA | NA | 163.0 |
| DLS | Z-Ave | F28 | 20150401-FT | 19.19 | NA | NA | 19.88 |
| | (d nm) | F29 | 20150402-FT | 19.34 | NA | NA | 19.96 |
| | | F30 | 20150403-FT | 14.49 | NA | NA | 14.51 |
| | | F31 | 20150404-FT | 14.68 | NA | NA | 14.87 |
| | PdI | F28 | 20150401-FT | 0.095 | NA | NA | 0.098 |
| | | F29 | 20150402-FT | 0.094 | NA | NA | 0.0 [symbol] |
| | | F30 | 20150403-FT | 0.056 | NA | NA | 0.075 |
| | | F31 | 20150404-FT | 0.066 | NA | NA | 0.077 |

[symbol]

indicates data missing or illegible when filed

The select formulations tested maintained stability when exposed to mechanical, thermal, and chemical stresses. Formulations buffered with sodium phosphate or sodium succinate exhibited similar stability in all stress conditions tested and either buffering systems would be appropriate.

### Example 3. Determine if Formulation Containing 25 mM Sodium Phosphate, 25 mM Sodium Chloride, 8.7% Trehalose, 0.01% PS 80 Buffered at pH 6.5 Provides Suitable Stability for 10 mg/ml and 30 mg/ml M281 Injection

To determine if formulations containing 25 mM sodium phosphate, 25 mM sodium chloride, 8.7% trehalose, 0.01% w/v PS80 buffered pH 6.5 provide suitable stability for both 10 mg/mL and 30 mg/mL M281 injection, properties of the formulations were assessed by analytical assays following exposure to thermal and shear stresses.

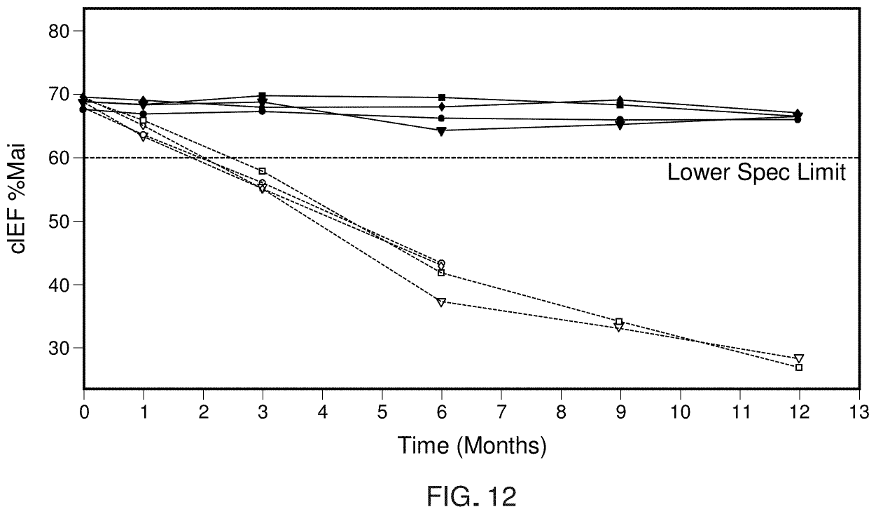

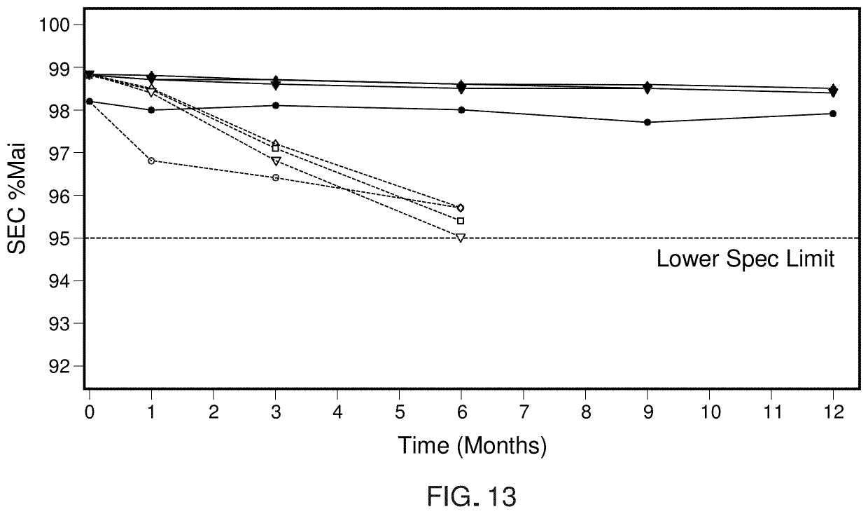

Formulations containing 25 mM sodium phosphate, 25 mM sodium chloride, 8.7% Trehalose, 0.01% w/v PS80 and either 10 mg/ml or 30 mg/ml M281 buffered pH 6.5 were prepared. A range of analytical assays were used to assess the product quality as part of these studies. Of the attributes evaluated, the most substantial changes over the course of the studies were observed in charge variants as measured by cIEF and aggregation levels as measured by SEC. Therefore, cIEF and SEC were selected as stability indicating assays. Charge variants by cIEF (FIG. 12) and soluble aggregates by SEC (FIG. 13) for drug product at 10 mg/mL (Lot E, Lot F, and Lot B) and 30 mg/mL (Lot D) were compared in long-term and accelerated stability studies. The rate of the main species degradation for M281 drug product by cIEF and SEC at 10 mg/mL and 30 mg/mL is comparable at both long term storage conditions (2 to 8° C.) and at accelerated storage conditions (25° C.).

Data from the forced degradation studies such as agitation, oxidation, thermal, and shear stress are shown in Table 10 through Table 13. The data shows similar degradation at both 10 mg/mL and 30 mg/mL formulations as measured by clEF and SEC assays.

TABLE 10
Comparison of % Main Species levels
under Agitation by SEC-HPLC and cIEF

| | |
|---|---|
| SEC-HPLC % Main | cIEF % Main |

| Agitation | 10 mg/ | 30 mg/ | 10 mg/ | 30 mg/ |
|---|---|---|---|---|
| (days) | mL Ab | mL Ab | mL Ab | mL Ab |
| 0 | 98.0 | 98.4 | 65.9 | 65.6 |
| 5 | 97.9 | 98.1 | NT | NT |
| 10 | 97.3 | 98.0 | 64.2 | 64.5 |

¹ cIEF = Capillary isoelectric focusing; NT = not tested; SEC-HPLC = size exclusion high performance liquid chromatography

TABLE 11
Comparison of % Main Species Levels
under Oxidation by SEC-HPLC and cIEF

| | |
|---|---|
| SEC-HPLC % Main | cIEF % Main |

| Oxidation | 10 mg/ | 30 mg/ | 10 mg/ | 30 mg/ |
|---|---|---|---|---|
| (hours) | mL Ab | mL Ab | mL Ab | mL Ab |
| 0 | 98.1 | 98.4 | 65.8 | 65.6 |
| 1 | 98.3 | 98.3 | NT | NT |
| 3 | 98.3 | 98.4 | NT | NT |
| 6 | 98.4 | 98.4 | 62.7 | 65.0 |

cIEF = Capillary isoelectric focusing;
NT = not tested;
SEC-HPLC = size exclusion high performance liquid chromatography

TABLE 12
Comparison of % Main Species Levels under Thermal
Stress at 40° C. by SEC-HPLC and cIEF.

| | |
|---|---|
| SEC-HPLC % Main | cIEF % Main |

| Thermal | 10 mg/ | 30 mg/ | 10 mg/ | 30 mg/ |
|---|---|---|---|---|
| (days) | mL Ab | mL Ab | mL Ab | mL Ab |
| 0 | 98.0 | 98.4 | 65.9 | 65.6 |
| 5 | 98.1 | 97.0 | NT | NT |
| 10 | 97.8 | 96.7 | 54.3 | 54.6 |

¹ cIEF = Capillary isoelectric focusing; NT = not tested; SEC-HPLC = size exclusion high performance liquid chromatography

TABLE 13
Comparison of % Main Species Levels under
Shear Stress by SEC-HPLC and cIEF

| | | |
|---|---|---|
| Shear | SEC % Main | cIEF % Main |

| Stress | 10 mg/ | 30 mg/ | 10 mg/ | 30 mg/ |
|---|---|---|---|---|
| (cycles) ² | mL Ab | mL Ab | mL Ab | mL of Ab |
| 0 | 98.4 | 98.2 | 68.7 | 67.2 |
| 1 | 98.4 | 98.3 | 68.4 | 67.2 |
| 5 | 98.3 | 98.2 | 67.9 | 67.4 |
| 10 | 98.3 | 98.2 | 68.0 | 66.6 |

¹ cIEF = Capillary isoelectric focusing; SEC-HPLC = size exclusion high performance liquid chromatography
² Cycles refer to the number of times M281 was recirculated through the filling pump to mimic worst case scenario

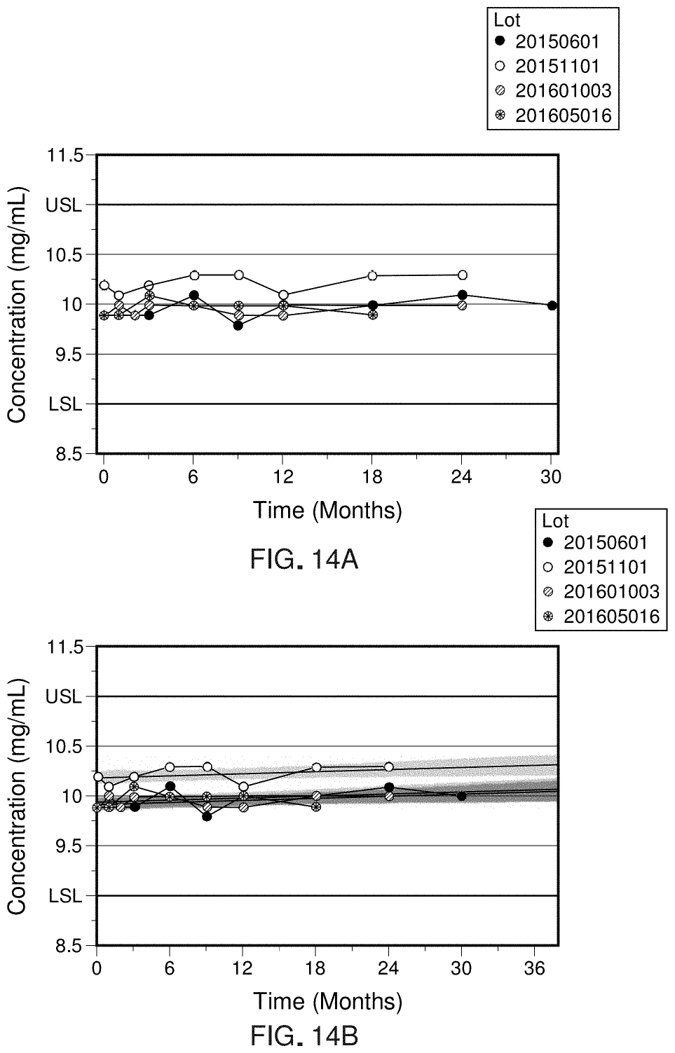

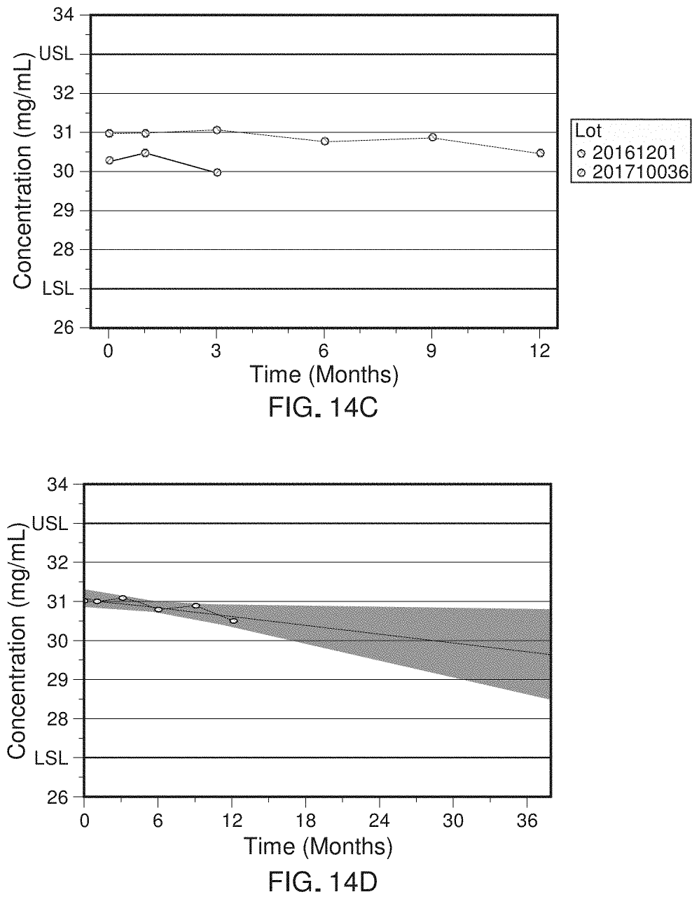

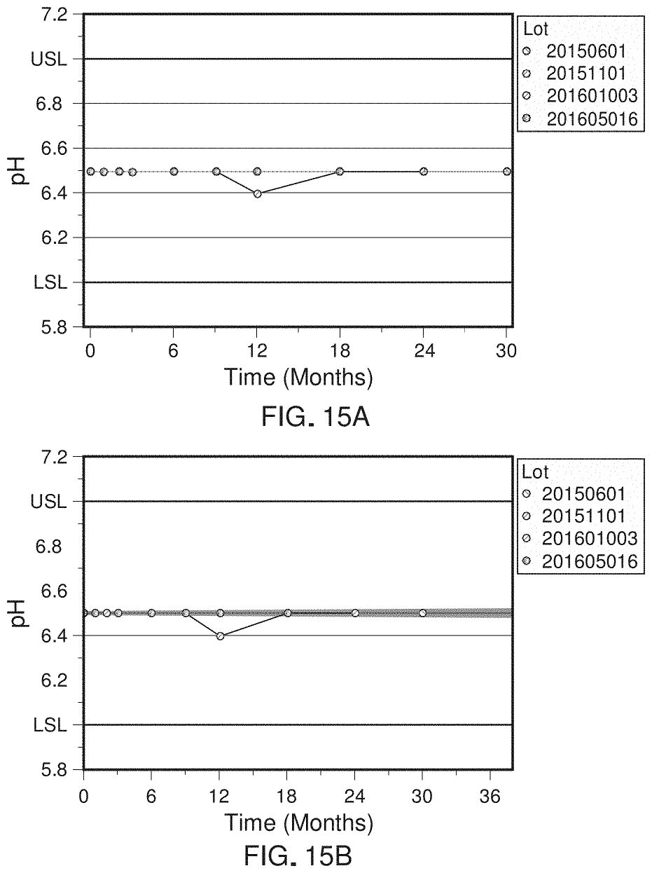

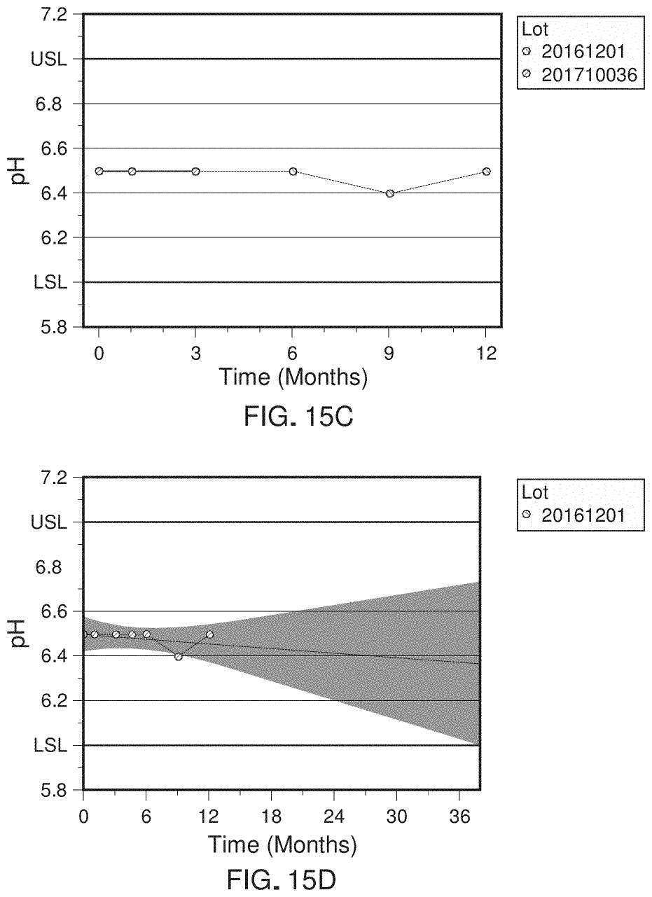

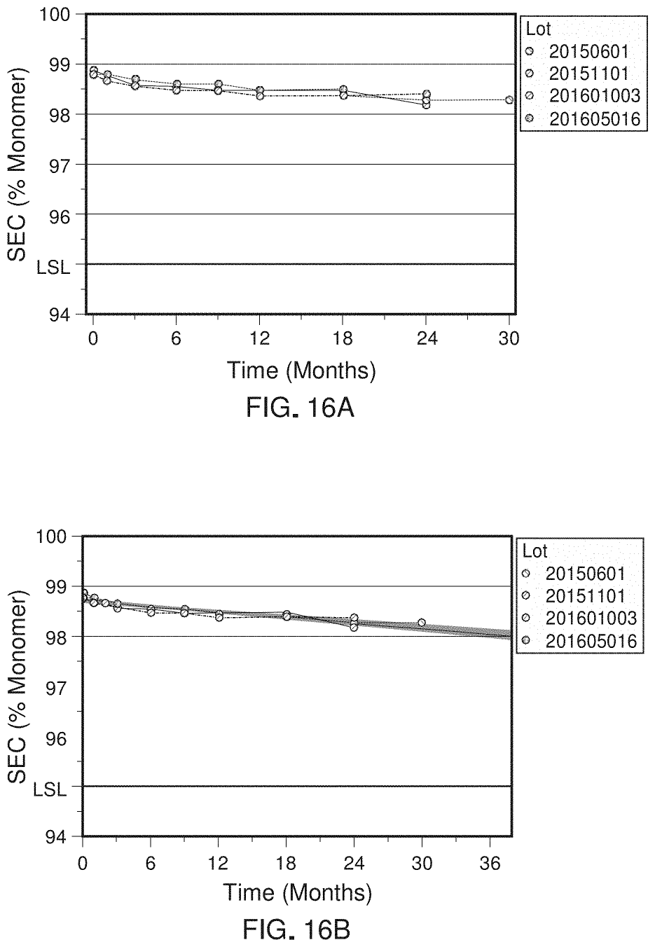

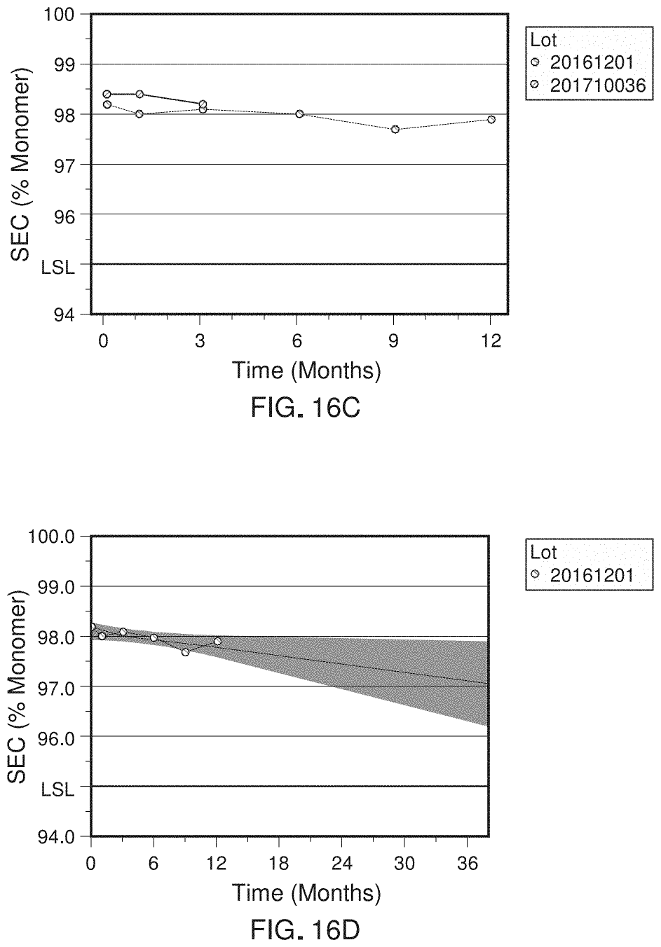

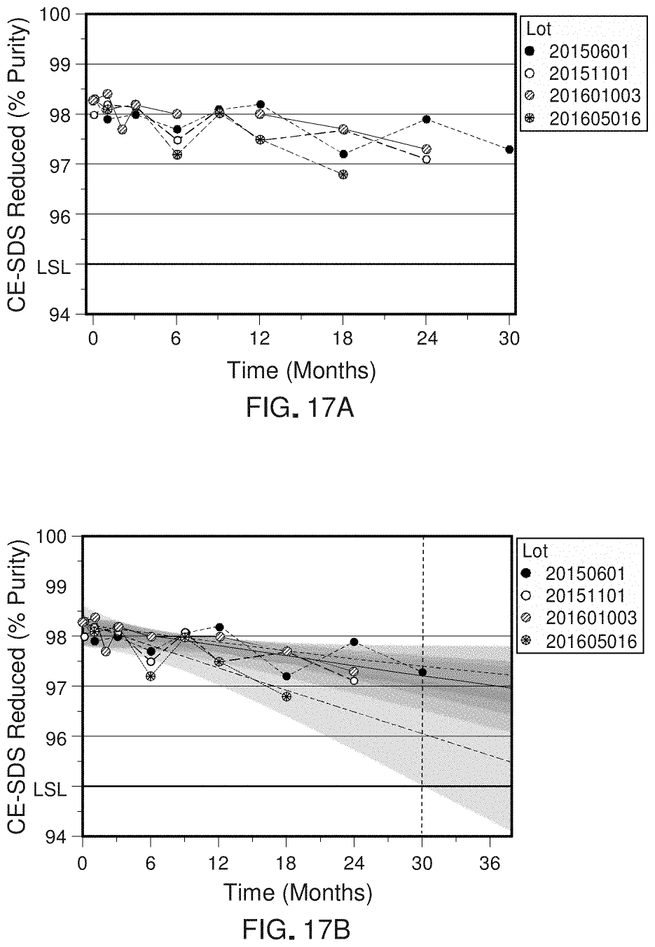

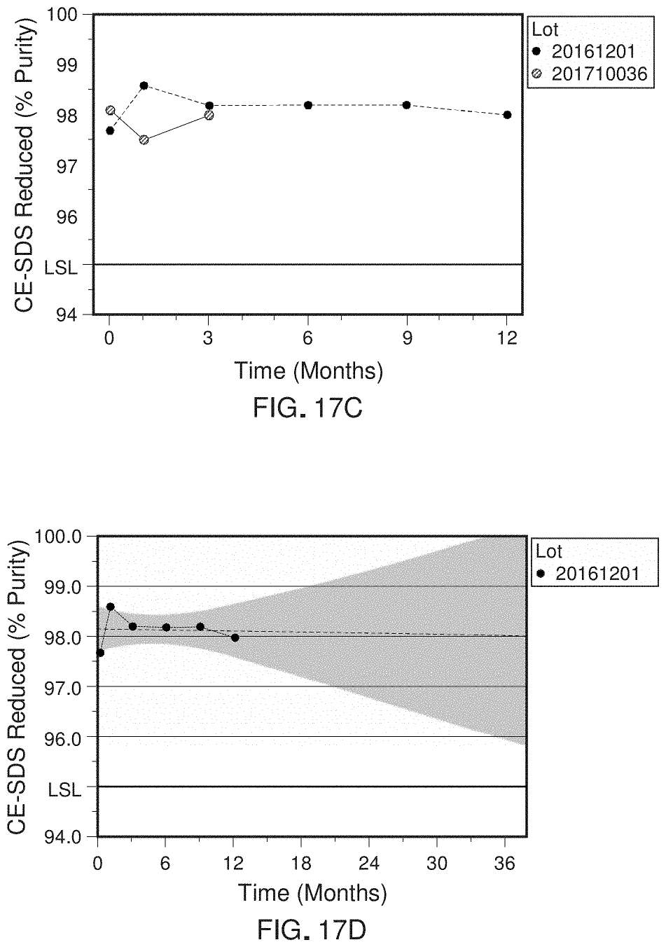

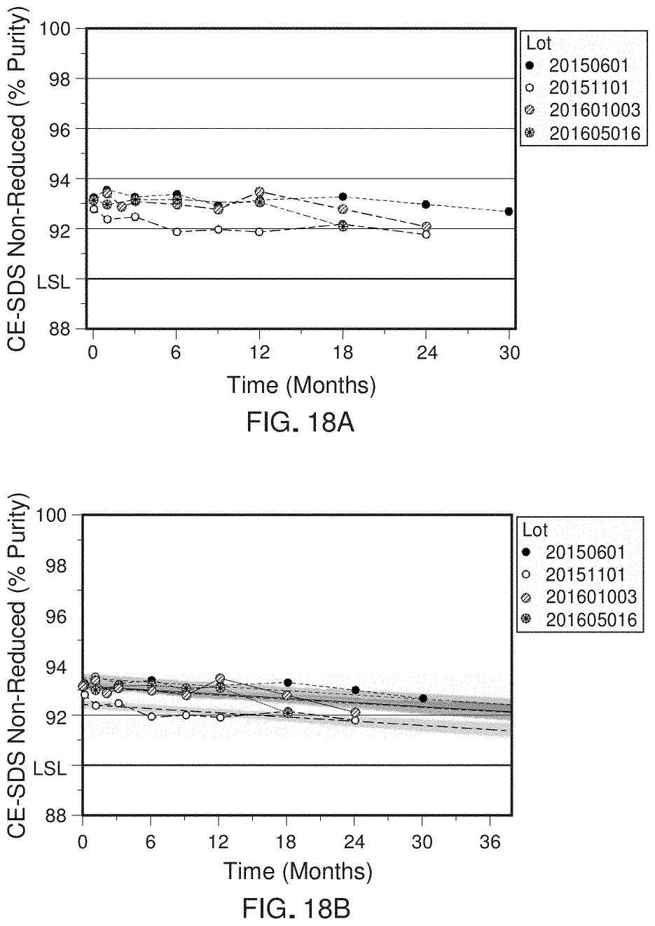

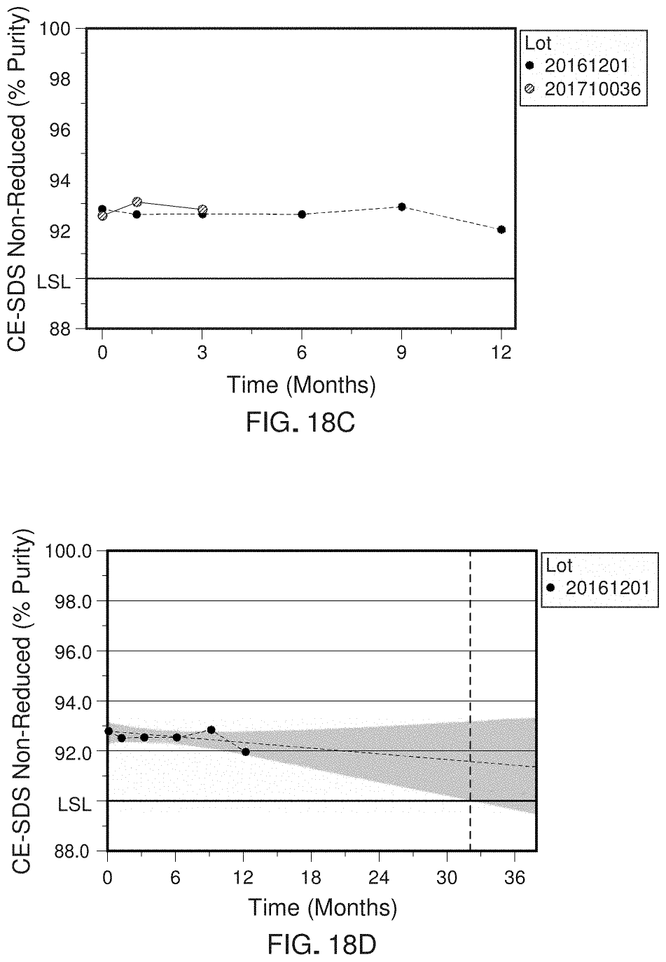

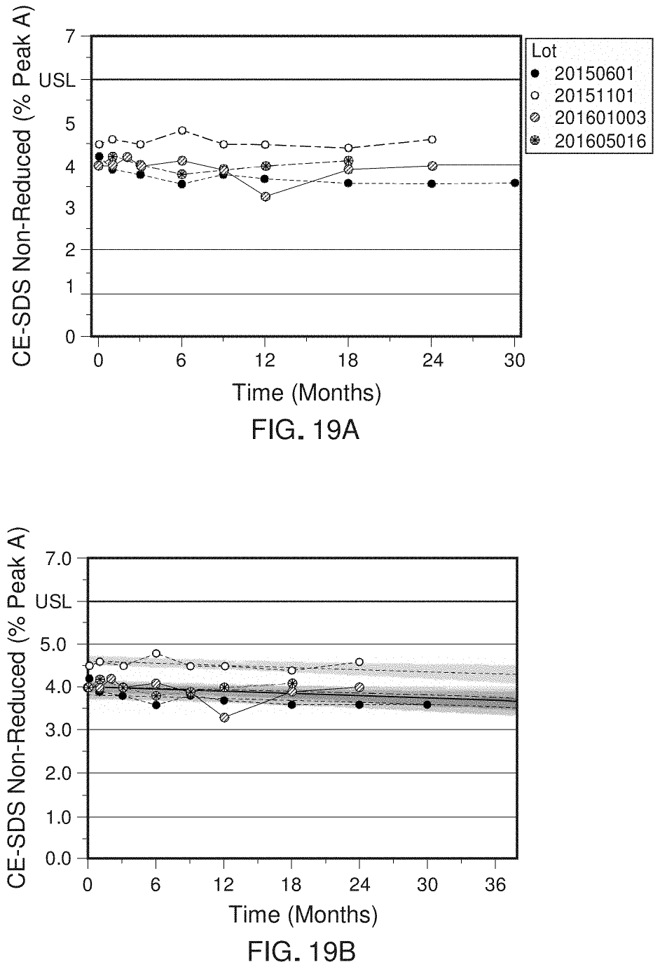

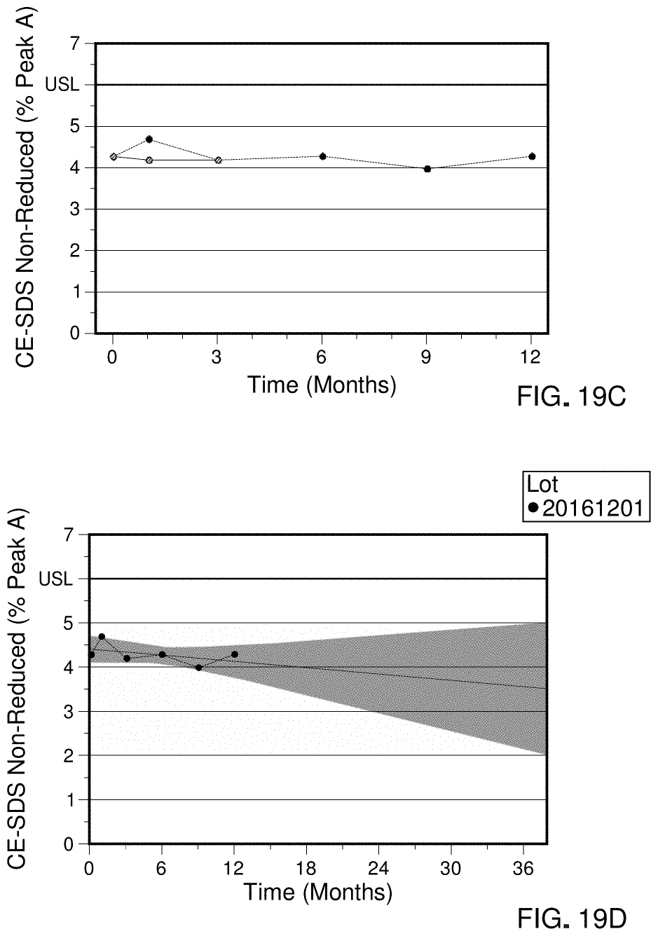

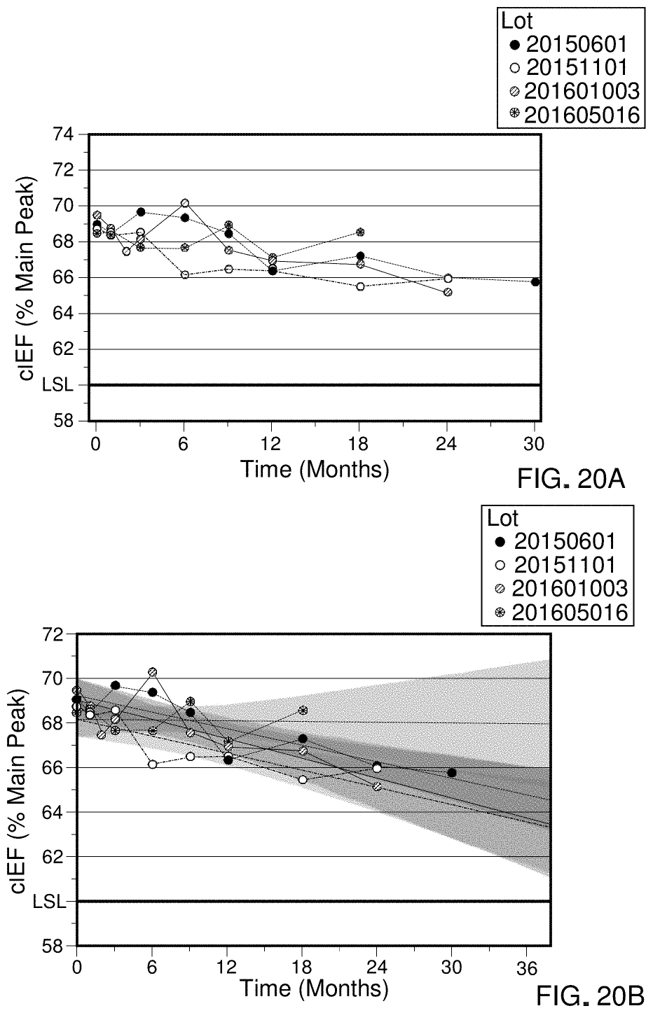

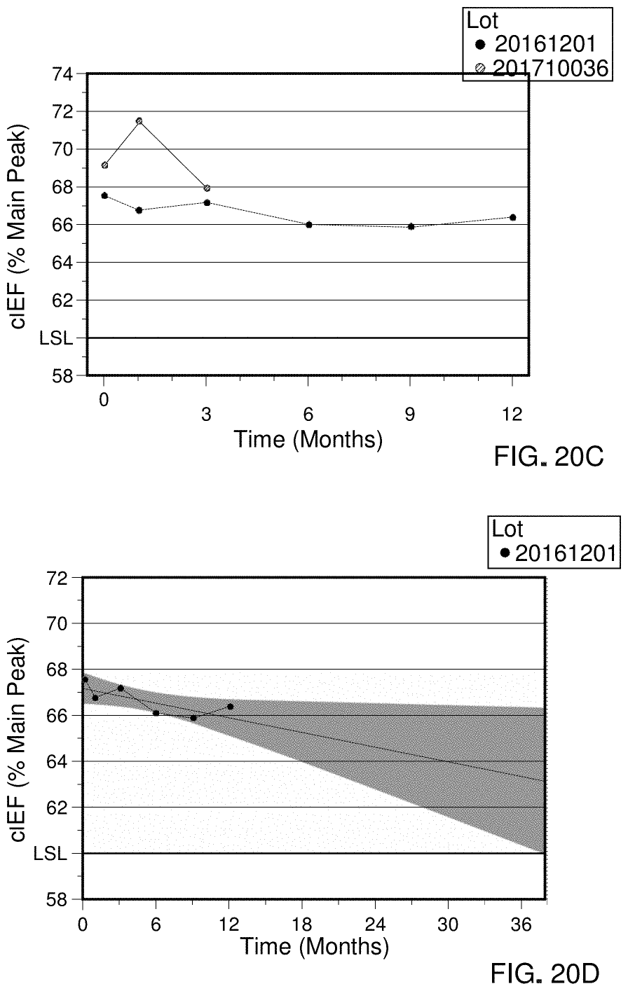

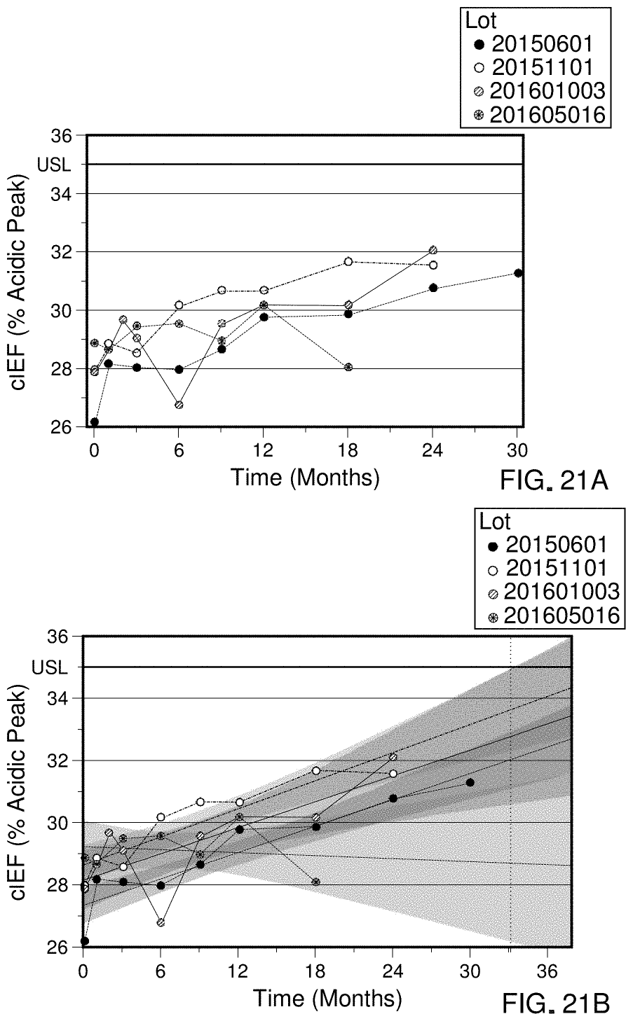

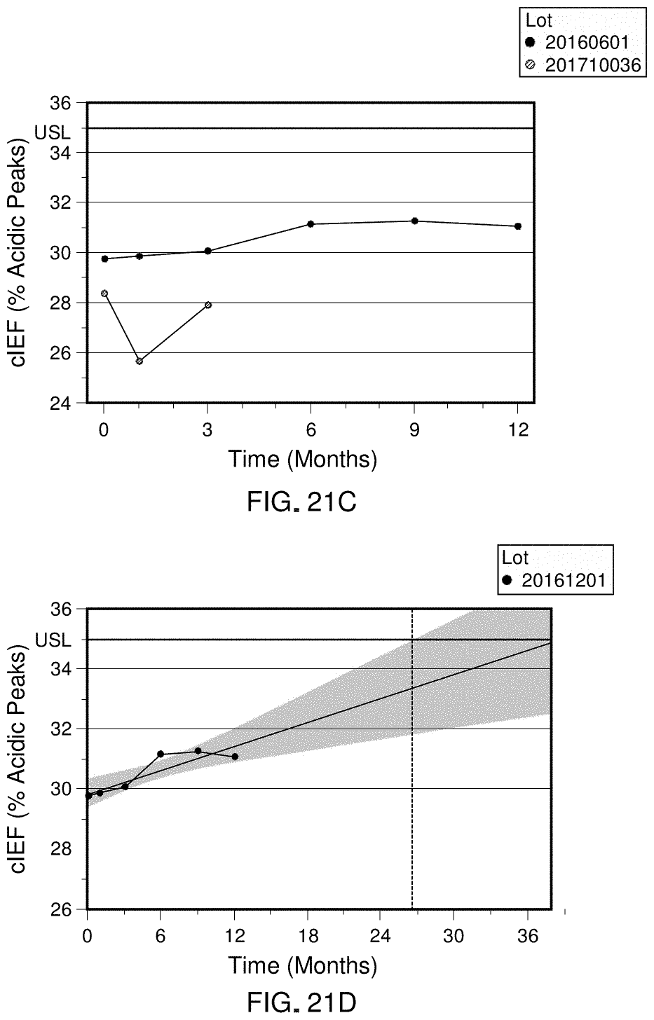

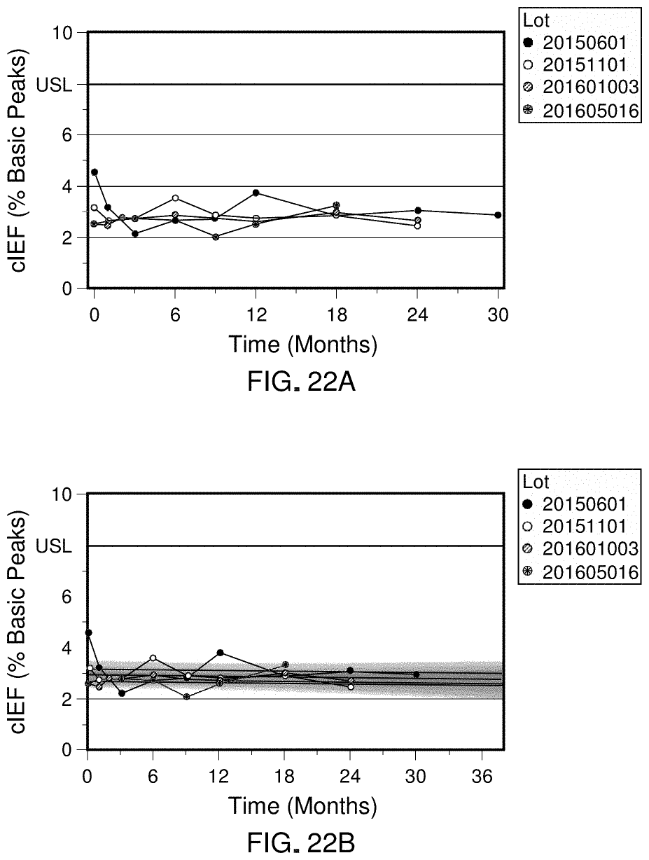

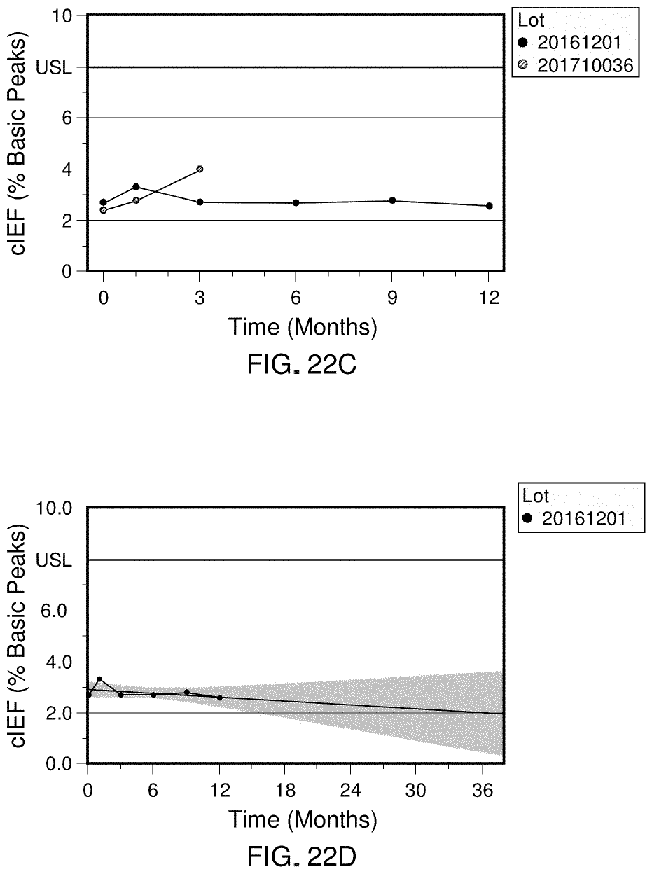

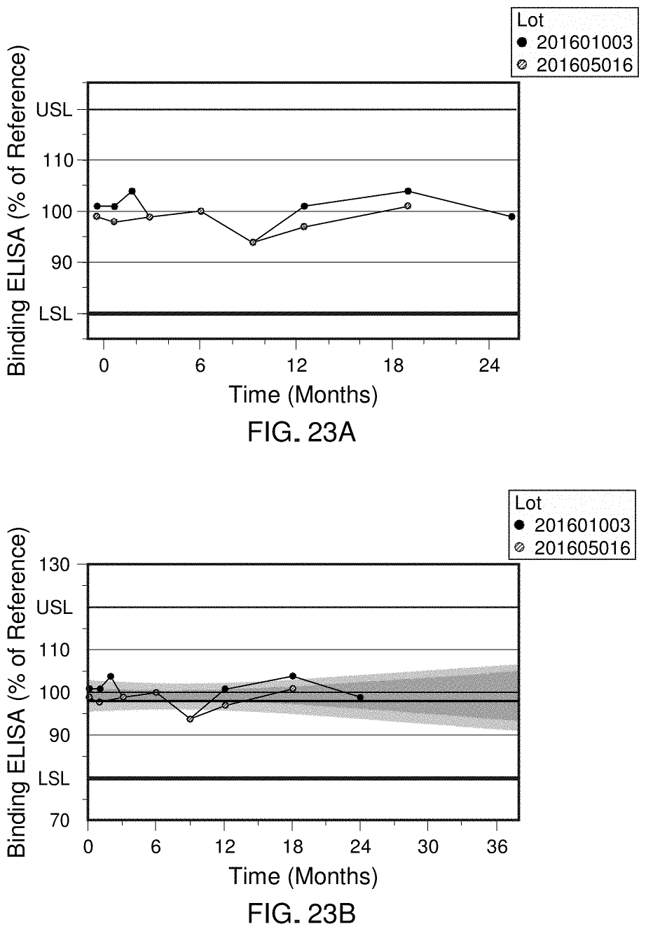

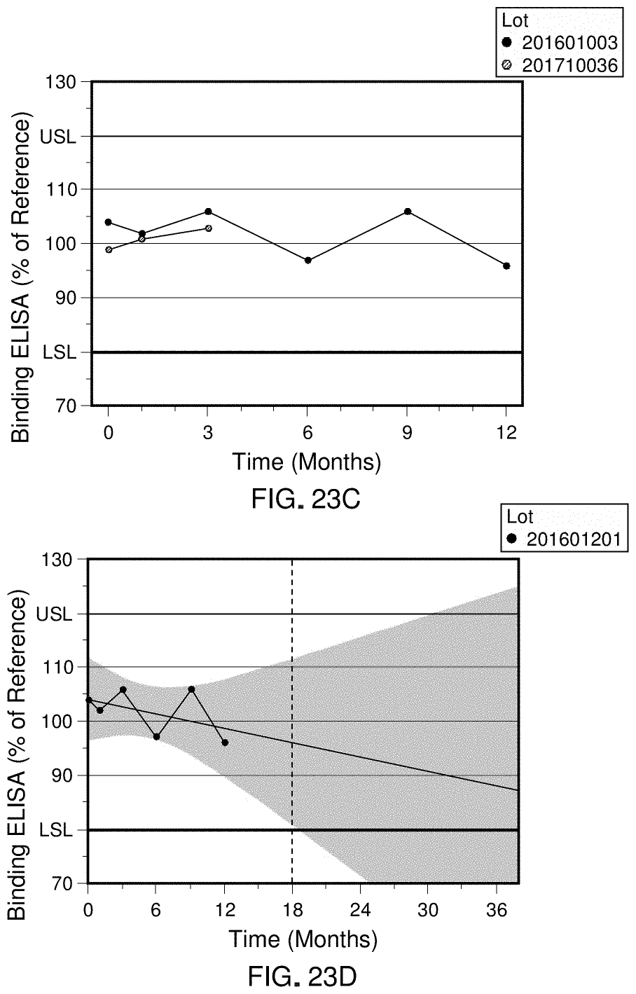

The results of degradation rates observed from forced degradation and stability data generated (FIGS. 14 to 23) indicate similar degradation rates for formulations containing 25 mM sodium phosphate, 25 mM sodium chloride, 8.7% Trehalose, 0.01% polysorbate 80 and either 10 or 30 mg/ml antibody buffered at pH 6.5. The data indicates that a formulation of 25 mM sodium phosphate, 25 mM sodium chloride, 8.7% w/w Trehalose, 0.01% w/v polysorbate 80, pH 6.5, provides stability at both 10 mg/mL and 30 mg/mL, up to 30 months and 18 months respectively.

The impact of higher levels of polysorbate 80 on sub-visible particles in both static and agitated samples was examined in a formulation that was contained the antibody, 25 mM sodium phosphate, 25 mM sodium chloride, 8.7% Trehalose, 0.01% PS 80 and either 10 or 30 mg/ml antibody buffered at pH 6.5. Sub-visible particles in the formulation samples were analyzed for their size and morphology using a FlowCAM particle imaging system. Briefly, aliquots of formulations were degassed for 30 minutes at 75 torr and 500 μL of each sample was injected into the analyzer. Real-time images of the particles in the fluid were captured as they passed through the flow cell. Total particle count enumerating all particles in the sample was collected and is presented in Table 14. Subtracted particle count for the formulations was generated by the application of a digital filter to the raw data to eliminate contributions due to non-proteinaceous repeating and circular particles (e.g., likely bubbles) and is presented in Table 15. In this analysis particles that are less than 5 μm are considered to be too small for prescise filtering or subtracting in particle imaging analysis.

TABLE 15
Impact of Polysorbate 80 on Sub-visible Particles (Raw Data)

| | | | | | 0.01% | 0.05% | 0.10% |
|---|---|---|---|---|---|---|---|
| | | 0.01% | 0.05% | 0.10% | PS80 | PS80 | PS80 |
| | Wa- | PS80 | PS80 | PS80 | Agita- | Agita- | Agita- |
| Size | ter | Static | Static | Static | tion | tion | tion |
| >2 μm | 18 | 10154 | 24210 | 2588 | 4658 | 2521 | 661 |
| >5 μm | 0 | 4787 | 10688 | 1053 | 1630 | 1270 | 249 |
| >10 μm | 0 | 1492 | 3563 | 351 | 474 | 298 | 57 |
| >25 μm | 0 | 210 | 476 | 110 | 36 | 10 | 10 |

TABLE 16
Impact of Polysorbate 80 on Sub-visible Particles (Subtractd Data)

| | | | | | 0.01% | 0.05% | 0.10% |
|---|---|---|---|---|---|---|---|
| | | 0.01% | 0.05% | 0.10% | PS80 | PS80 | PS80 |
| | Wa- | PS80 | PS80 | PS80 | Agita- | Agita- | Agita- |
| Size | ter | Static | Static | Static | tion | tion | tion |
| >5 μm | 0 | 4787 | 10688 | 1053 | 1569 | 1184 | 192 |
| >10 μm | 0 | 1492 | 3563 | 351 | 474 | 298 | 57 |
| >25 μm | 0 | 210 | 476 | 110 | 36 | 10 | 10 |

1. A pharmaceutical composition comprising an antibody at a concentration of 10 or 30 mg/ml, 20-30 mM sodium phosphate, 20-30 mM sodium chloride, 80-100 mg/ml Trehalose, and 0.10-0.005% w/v Polysorbate 80, buffered at pH 6.5,
wherein the antibody comprises a heavy chain comprising the amino acid sequence of SEQ ID NO:2 with up to 5 single amino acid insertions, substitutions or deletions and a light chain comprising the amino acid sequence of SEQ ID NO: 1 with up to 5 single amino acid insertions, substitutions or deletions.

2. The pharmaceutical composition of claim 1, wherein the pharmaceutical composition comprises 25 mM sodium phosphate.

3. The pharmaceutical composition of claim 1, wherein the pharmaceutical composition comprises 25 mM sodium chloride.

4. The pharmaceutical composition of claim 1, wherein the pharmaceutical composition comprises 90-91 mg/ml Trehalose.

5. The pharmaceutical composition of claim 1, wherein the pharmaceutical composition comprises 90.5 mg/ml Trehalose.

6. The pharmaceutical composition of claim 1, wherein the pharmaceutical composition comprises 0.01% w/v Polysorbate 80.

7. The pharmaceutical composition of claim 1, wherein the pharmaceutical composition comprises 25 mM sodium phosphate, 25 mM sodium chloride, 90.5 mg/ml Trehalose, and 0.01% Polysorbate 80.

8. The pharmaceutical composition of claim 1, wherein the composition does not comprise any additional excipients.

9. The pharmaceutical composition of claim 1, wherein the antibody comprises a heavy chain comprising the amino acid sequence of SEQ ID NO:2 with up to 2 single amino acid insertions, substitutions or deletions and having a light chain comprising the amino acid sequence of SEQ ID NO: 1 with up to 2 single amino acid insertions, substitutions or deletions.

10. The pharmaceutical composition of claim 1, wherein the antibody comprises a heavy chain comprising the amino acid sequence of SEQ ID NO:2 with up to 2 single amino acid substitutions and having a light chain comprising the amino acid sequence of SEQ ID NO: 1 with up to 2 single amino acid substitutions.

11. The pharmaceutical composition of claim 1, wherein the antibody comprises a heavy chain comprising the amino acid sequence of SEQ ID NO:2 and having a light chain comprising the amino acid sequence of SEQ ID NO:1.

12. A pharmaceutical composition comprising an antibody at a concentration of 10 or 30 mg/ml, 20-30 mM sodium succinate, 20-30 mM sodium chloride, 89-92 mg/ml Trehalose, and 0.1-0.005% w/v Polysorbate 80, buffered at pH 6.5,
wherein the antibody comprises a heavy chain comprising the amino acid sequence of SEQ ID NO:2 with up to 5 single amino acid insertions, substitutions or deletions and a light chain comprising the amino acid sequence of SEQ ID NO: 1 with up to 5 single amino acid insertions, substitutions or deletions.

13. The pharmaceutical composition of claim 12, wherein the pharmaceutical composition comprises 25 mM sodium succinate.

14. The pharmaceutical composition of claim 12, wherein the pharmaceutical composition comprises 25 mM sodium chloride.

15. The pharmaceutical composition of claim 12, wherein the pharmaceutical composition comprises 90-91 mg/ml Trehalose.

16. The pharmaceutical composition of claim 12, wherein the pharmaceutical composition comprises 90.5 mg/ml Trehalose.

17. The pharmaceutical composition of claim 12, wherein the pharmaceutical composition comprises 0.01% w/v Polysorbate 80.

18. The pharmaceutical composition of claim 12, wherein the pharmaceutical composition comprises 25 mM sodium succinate, 25 mM sodium chloride, 90.5 mg/ml Trehalose, and 0.01% Polysorbate 80.

19. The pharmaceutical composition of claim 12, wherein the composition does not comprise any additional excipients.

20. The pharmaceutical composition of claim 12, wherein the antibody comprises a heavy chain comprising the amino acid sequence of SEQ ID NO:2 with up to 2 single amino acid insertions, substitutions or deletions and having a light chain comprising the amino acid sequence of SEQ ID NO: 1 with up to 2 single amino acid insertions, substitutions or deletions.

21. The pharmaceutical composition of claim 12, wherein the antibody comprises a heavy chain comprising the amino acid sequence of SEQ ID NO:2 with up to 2 single amino acid substitutions and having a light chain comprising the amino acid sequence of SEQ ID NO: 1 with up to 2 single amino acid substitutions.

22. The pharmaceutical composition of claim 12, wherein the antibody comprises a heavy chain comprising the amino acid sequence of SEQ ID NO:2 and having a light chain comprising the amino acid sequence of SEQ ID NO: 1.

23. The pharmaceutical composition of claim 12, wherein the antibody comprises a heavy chain consisting of the amino acid sequence of SEQ ID NO:2 and having a light chain consisting of the amino acid sequence of SEQ ID NO: 1.

24. The pharmaceutical composition of claim 1, wherein the composition does not comprise any polysorbates other than polysorbate 80.

25. The pharmaceutical composition of claim 1, wherein the composition does not comprise include any polymers other than a polysorbate, or polysorbate 80.

26. The pharmaceutical composition of claim 12, wherein the composition does not comprise include any polymers other than polysorbate, and/or wherein the composition does not comprise any polysorbates other than polysorbate 80.
